(19) United States
(12) Patent Application Publication
(10) Pub. No.: US 2025/0161211 A1
(43) Pub. Date: May 22, 2025

(54) LIQUID FORMULATION COMPRISING A FUSION PROTEIN INCLUDING a-GALACTOSIDASE A

(71) Applicants: GREEN CROSS CORPORATION, Yongin-si, GYEONGGI-DO (KR); HANMI PHARM. CO., LTD., Hwaseong-si, GYEONGGI-DO (KR)

(72) Inventors: Miroo KIM, Yongin-si, Gyeonggi-do (KR); Miri YOO, Yongin-si, Gyeonggi-do (KR); Jae Woon SON, Yongin-si, Gyeonggi-do (KR); Da-eui PARK, Yongin-si, Gyeonggi-do (KR); Shin-Ae YI, Yongin-si, Gyeonggi-do (KR); Jin Young KIM, Hwaseong-si, Gyeonggi-do (KR); Jun Sub PARK, Hwaseong-si, Gyeonggi-do (KR); Doo Seo JANG, Hwaseong-si, Gyeonggi-do (KR); Sung Hee HONG, Hwaseong-si, Gyeonggi-do (KR); Sang Yun KIM, Hwaseong-si, Gyeonggi-do (KR)

(21) Appl. No.: 18/832,725

(22) PCT Filed: Mar. 10, 2023
(86) PCT No.: PCT/KR2023/003299

§ 371 Date: Jul. 24, 2024

(30) Foreign Application Priority Data
Mar. 15, 2022 (KR) 10-2022-0032090

(51) Int. Cl. A61K 9/08; A61K 9/00; A61K 38/47; A61K 47/02; A61K 47/18; A61K 47/22; A61K 47/26; A61K 47/68

(52) U.S. Cl. CPC A61K 9/08; A61K 9/0019; A61K 38/47; A61K 47/02; A61K 47/183; A61K 47/22; A61K 47/26; A61K 47/6815; C12Y 302/01022

(57) ABSTRACT

The present invention relates to a liquid formulation including a fusion protein of α-galactosidase A and a preparation method thereof, wherein the liquid formulation not only has storage stability by including a composition providing structural stability to a fusion protein of α-galactosidase A, but also has excellent stability although the fusion protein is contained at a high concentration.

### TECHNICAL FIELD

The present invention relates to a liquid formulation including a fusion protein of α-galactosidase A and a preparation method thereof, and a use thereof.

### BACKGROUND ART

Lysosomes are intracellular organelles involved in degradation of proteins, various lipids such as glycolipids and cholesterol, and carbohydrates and recycling of degraded products as primary constituents of new proteins, membrane components, and other molecules. Diseases associated with functions thereof include lysosomal storage diseases (LSDs).

Lysosomal Storage Diseases (LSDs) are diseases that result from defects in genes encoding enzymes present in lysosomes and degrading glycolipid or polysaccharide waste products, and biological activity of lysosomal enzymes is significantly reduced or absent in cells and tissues of individuals with lysosomal storage diseases. Such deficiency of degradative enzymes causes accumulation of substances in large quantities, thereby causing a problem in the function of cells.

Fabry disease, known as a lysosomal storage disease, is an inherited disorder of glycolipid (glycosphingolipid) metabolism resulting from deficient or insufficient activity of alpha-galactosidase A, which is a hydrolase present in lysosomes. Fabry disease, as a representative disease of α-galactosidase A deficiency, is a disease associated with X chromosome, and typical Fabry patients generally have an α-galactosidase A activity of less than 1% compared to that of normal people and have a wide range of symptoms including severe pain in the extremities (acroparesthesia), hypohidrosis, corneal and lenticular changes, skin lesions (angiokeratoma), renal failure, cardiovascular disease, pulmonary failure, nervous system symptoms, and stroke.

Fabry disease causes progressive accumulation of globotriaosylceramide (Gb3) in most tissues of the body. The accumulation of Gb3 is mainly found in vascular endothelia. Such progressive accumulation of Gb3 in vascular endothelia leads to ischemia and infarction in organs such as the kidneys, heart, or brain.

A number of studies have been conducted into enzyme-replacement therapy (ERT) as a representative method for treating lysosomal storage disease (Frances M. Platt et al., J Cell Biol. 2012 Nov. 26; 199(5): 723-34). In particular, since lysosomal storage diseases are caused by genetic defects in particular enzymes, a therapy replacing the deficient enzyme is essential. Enzyme-replacement therapy is a standard therapy in lysosomal storage diseases and may have an effect on alleviating existing symptoms or delaying the progress of the disease by replacing the deficient enzyme. Accordingly, studies have been conducted on various formulations including α-galactosidase A for preventing and treating α-galactosidase A deficiency.

However, in the case of formulations, a composition of a formulation for obtaining stability significantly varies according to a structure, physicochemical properties, dosage, etc., of an active ingredient (e.g., protein drug), and thus it is essential to develop a particular composition suitable for the active ingredient, and development of a formulation including α-galactosidase A is insufficient. Particularly, in the case of a liquid formulation including an α-galactosidase A fusion protein at a high concentration, storage stability considerably deteriorates due to a problem such as protein precipitation, and thus conventional liquid formulations include α-galactosidase A fusion protein at a concentration of 1 mg/ml or less. However, there is a need to develop a highly stable formulation including a high-concentration α-galactosidase A fusion protein to increase efficacy.

### DISCLOSURE OF INVENTION

### Technical Problem

Development of a stable liquid formulation, suitable for long-term storage and having the α-galactosidase A activity for a long time, is insufficient, and a liquid formulation including an α-galactosidase A fusion protein at a high concentration has not been developed, and thus there is still a need to develop a liquid formulation including a novel composition of an α-galactosidase A fusion protein.

### Solution to Problem

An object of the present invention is to provide a liquid formulation including a fusion protein of α-galactosidase A.

Another object of the present invention is to provide a method for preparing the liquid formulation.

### Advantageous Effects of Invention

The liquid formulation according to the present invention not only has storage stability by including a composition providing structural stability to a fusion protein of α-galactosidase A, but also has excellent stability although the fusion protein is contained at a high concentration.

### BRIEF DESCRIPTION OF DRAWINGS

FIG. 1 shows measurement results of Tm and Tagg values according to pH change.

FIG. 2 shows the Tm values identified in FIG. 1.

FIG. 3 shows the Tagg values identified in FIG. 1.

FIG. 4 shows SE-HPLC analysis results of soluble aggregates during room temperature storage according to pH change of a liquid formulation.

FIG. 5 shows SE-HPLC analysis results of fragments during room temperature storage according to pH change of a liquid formulation.

FIG. 6 shows SE-HPLC analysis results of recovery of native form during room temperature storage according to pH change of a liquid formulation.

FIG. 7 shows SE-HPLC analysis results of soluble aggregates during cold storage according to pH change of a liquid formulation.

FIG. 8 shows SE-HPLC analysis results of fragments during cold storage according to pH change of a liquid formulation.

FIG. 9 shows SE-HPLC analysis results of recovery of native form during cold storage according to pH change of a liquid formulation.

FIG. 10 shows SE-HPLC analysis (FIG. 10(a)) and biological activity analysis (FIG. 10(b)) results of an α-galactosidase A fusion protein of liquid formulations with pH 6.0 and pH 8.0.

FIG. 11 shows SE-HPLC analysis results of soluble aggregates according to concentration of an isotonic agent of a liquid formulation.

FIG. 12 shows SE-HPLC analysis results of fragments according to concentration of an isotonic agent of a liquid formulation.

FIG. 13 shows SE-HPLC analysis results of recovery of native form according to concentration of an isotonic agent of a liquid formulation.

FIG. 14 shows SE-HPLC analysis results of accelerated stability according to type of an amino acid of a liquid formulation.

FIG. 15 shows measurement results of Tm values according to concentration of an amino acid.

FIG. 16 shows SE-HPLC analysis results of soluble aggregates according to concentration of an amino acid of a liquid formulation.

FIG. 17 shows SE-HPLC analysis results of recovery of native form according to concentration of an amino acid of a liquid formulation.

FIG. 18 shows SE-HPLC analysis results of soluble aggregates according to concentration of an amino acid of a liquid formulation including a high-concentration (50 mg/mL) of a fusion protein.

FIG. 19 shows SE-HPLC analysis results of recovery of native form according to concentration of an amino acid of a liquid formulation including a high-concentration (50 mg/ml) of a fusion protein.

FIG. 20 shows SE-HPLC chromatogram of a liquid formulation including a high-concentration (50 mg/mL) of a fusion protein.

FIG. 21 shows SE-HPLC analysis results of HMW1 and HMW2 aggregates in a liquid formulation including a high-concentration (50 mg/ml) of a fusion protein.

FIG. 22 shows appearances of a liquid formulation including a high-concentration (90 mg/ml) of a fusion protein.

FIG. 23 shows turbidity of a liquid formulation including a high-concentration (90 mg/ml) of a fusion protein.

### BEST MODE FOR CARRYING OUT THE INVENTION

An aspect of the present invention provides a liquid formulation including an α-galactosidase A fusion protein.

In a specific embodiment, the liquid formulation is characterized by including a fusion protein in which α-galactosidase A is linked to an immunoglobulin Fc region; a buffer; an isotonic agent; and an amino acid.

In another specific embodiment, the liquid formulation is characterized by including the fusion protein at a concentration of 10 mg/mL to 90 mg/mL; a buffer for maintaining a pH of the liquid formulation in a range of 5.0 to 6.5; and an amino acid at a concentration of 0.1% (w/v) to 4.0% (w/v).

The liquid formulation according to any one of the specific embodiments is characterized by including the fusion protein at a concentration of 10 mg/mL to 90 mg/ml; a buffer for maintaining a pH of the liquid formulation in a range of 5.5 to 6.5; and an amino acid at a concentration of 0.5% (w/v) to 4.0% (w/v).

The liquid formulation according to any one of the specific embodiments is characterized by including the fusion protein at a concentration of 10 mg/mL to 90 mg/ml; 10 mM to 50 mM histidine; and 1.0% (w/v) to 4.0% (w/v) serine.

The liquid formulation according to any one of the specific embodiments is characterized by including the fusion protein at a concentration of 10 mg/ml to 90 mg/mL; 10 mM to 50 mM histidine; 50 mM to 200 mM sodium chloride; and 1.0 to 4.0% (w/v) serine.

The liquid formulation according to any one of the specific embodiments is characterized in that the buffer includes histidine or a salt thereof, citric acid or a salt thereof, acetic acid or a salt thereof, phosphoric acid or a salt thereof, or any combination thereof.

The liquid formulation according to any one of the specific embodiments is characterized by further including an isotonic agent.

The liquid formulation according to any one of the specific embodiments is characterized in that the isotonic agent is sodium chloride.

The liquid formulation according to any one of the specific embodiments is characterized in that the isotonic agent is contained at a concentration of 50 mM to 200 mM.

The liquid formulation according to any one of the specific embodiments is characterized in that the amino acid is selected from the group consisting of arginine, serine, threonine, glutamine, glycine, alanine, and any combination thereof.

The liquid formulation according to any one of the specific embodiments is characterized in that the liquid formulation further comprises a non-ionic surfactant at a concentration of 0.005% (w/v) to 0.1% (w/v).

The liquid formulation according to any one of the specific embodiments is characterized in that the non-ionic surfactant is selected from the group consisting of poloxamer 188, polysorbate 20, polysorbate 40, polysorbate 60, polysorbate 80, and any combination thereof.

The liquid formulation according to any one of the specific embodiments is characterized in that the α-galactosidase A includes an amino acid sequence of SEQ ID NO: 1.

The liquid formulation according to any one of the specific embodiments is characterized in that the immunoglobulin Fc region includes an amino acid sequence of SEQ ID NO: 3.

The liquid formulation according to any one of the specific embodiments is characterized in that the α-galactosidase A fusion protein includes an amino acid sequence of SEQ ID NO: 4.

The liquid formulation according to any one of the specific embodiments is characterized in that the immunoglobulin Fc region is derived from IgG4.

The liquid formulation according to any one of the specific embodiments is characterized in that the immunoglobulin Fc region is aglycosylated.

The liquid formulation according to any one of the specific embodiments is characterized in that the fusion protein has a structure in which two molecules of α-galactosidase A are linked to each monomer of an immunoglobulin Fc region in a dimeric form.

The liquid formulation according to any one of the specific embodiments is characterized in that the liquid formulation is used to prevent or treat Fabry disease.

The liquid formulation according to any one of the specific embodiments is characterized in that the liquid formulation is administered via a subcutaneous route.

Another aspect of the present invention provides a method for preparing the liquid formulation.

In a specific embodiment, the method for preparing the liquid formulation includes mixing the fusion protein of α-galactosidase A; a buffer; and an amino acid.

In another specific embodiment, the method for preparing the liquid formulation includes further mixing the liquid formulation with an isotonic agent, a non-ionic surfactant, a preservative, or any combination thereof.

### MODE FOR THE INVENTION

Hereinafter, the present invention will be described in more detail.

Meanwhile, each of the descriptions and embodiments disclosed herein may be applied herein to describe different descriptions and embodiments. That is, all of the combinations of various factors disclosed herein belong to the scope of the present invention. Furthermore, the scope of the present invention should not be limited by the detailed descriptions provided hereinbelow.

Those skilled in the art will recognize or be able to ascertain, using no more than routine experimentation, many equivalents to specific embodiments of the present invention. Such equivalents are intended to be encompassed in the scope of the following claims.

Throughout the specification, not only the conventional one-letter and three-letter codes for naturally occurring amino acids, but also those three-letter codes generally allowed for other amino acids, such as Aib (2-aminoisobutyric acid)), Sar (N-methylglycine), and α-methyl-glutamic acid are used. In addition, the amino acids mentioned herein are abbreviated according to the nomenclature rules of IUPAC-IUB as follows.

| | |
|---|---|
| alanine Ala, A | arginine Arg, R |
| asparagine Asn, N | aspartic acid Asp, D |
| cysteine Cys, C | glutamic acid Glu, E |
| glutamine Gln, Q | glycine Gly, G |
| histidine His, H | isoleucine Ile, I |
| leucine Leu, L | lysine Lys, K |
| methionine Met, M | phenylalanine Phe, F |
| proline Pro, P | serine Ser, S |
| threonine Thr, T | tryptophan Trp, W |
| tyrosine Tyr, Y | valine Val, V |

An aspect of the present invention provides a liquid formulation including a fusion protein in which α-galactosidase A is linked to an immunoglobulin Fc region.

Specifically, the present invention relates to a liquid formulation including a fusion protein in which α-galactosidase A is linked to an immunoglobulin Fc region in a pharmacologically effective amount, a buffer, and an amino acid. In addition, the liquid formulation of the present invention may further include an isotonic agent, a non-ionic surfactant, a preservative, or any combination thereof, without being limited thereto.

As used herein, the term “liquid formulation” refers to a drug formulated in a liquid form and includes both a liquid formulation for internal use and a liquid formulation for external use.

The liquid formulation of the present invention includes a substance that stably maintain and/or store the α-galactosidase A fusion protein having the pharmacological efficacy for a certain period of time while the α-galactosidase A fusion protein is formulated in a liquid phase. A component contained in the liquid formulation in addition to the α-galactosidase A fusion protein having the pharmacological efficacy may be interchangeably used with stabilizer. As used herein, the term “stabilizer” refers to a substance stably maintaining a component of a formulation such as an active ingredient for a certain period of time.

The liquid formulation of the present invention is characterized by including a composition of a stabilizer capable of stabilizing a structure of the fusion protein such that the pharmacological efficacy of the α-galactosidase A fusion protein is maintained even after long-term storage.

The α-galactosidase A fusion protein of the present invention has a structure in which α-galactosidase A is fused to an immunoglobulin Fc region and an unfolding phenomenon, in which the structure of the protein is unfolded by various external factors (e.g., pH, temperature, osmosis, and presence of stabilizer), causes loss of enzymatic activity resulting in a significant decrease in the pharmacological efficacy of the formulation including α-galactosidase. Particularly, α-galactosidase A sensitively reacts with the pH environment such that one of the two domains constituting α-galactosidase A is first unfolded (to form soluble aggregates) and the other domain of α-galactosidase A and the immunoglobulin Fc region are sequentially unfolded to form higher-order aggregates and finally, unfolding of CH2 and CH3 domains of the immunoglobulin Fc region (to form insoluble aggregates) causes precipitates recognizable by visual observation. The structure of the α-galactosidase A fusion protein may not be maintained due to a stress generated while the liquid formulation is stored, and the risk of immunogenicity increases when soluble aggregates or higher-order aggregates are formed, causing a safety problem.

The present inventors have found not only that including a composition inhibiting unfolding of the α-galactosidase A fusion protein is important for stability of the liquid formulation, but also that the enzymatic activity of α-galactosidase A is maintained since the structure of an active site is retained until insoluble aggregates are formed even after α-galactosidase A is unfolded and soluble aggregates are formed therefrom, and identified that it is important to adjust the pH and add a stabilizer (e.g., amino acid) to maintain the structural stability of the α-galactosidase A fusion protein as much as possible and furthermore, to prevent formation of insoluble aggregates caused as the unfolding phenomenon of the soluble aggregates proceeds, thereby completing the present invention. The liquid formulation of the present invention may have the structure and activity of the fusion protein maintained even after long-term storage and have high medication safety due to low immunogenicity.

In a specific example, the liquid formulation according to the present invention may include: a fusion protein in which α-galactosidase A is linked to an immunoglobulin Fc region; a buffer; and an amino acid, more specifically, include: a 10 mg/mL to 90 mg/mL fusion protein; a buffer; and a 0.1% (w/v) to 4.0% (w/v) amino acid, wherein a pH is from 5.0 to 6.5.

Additionally, in another example, the liquid formulation may include: a 10 mg/mL to 90 mg/ml fusion protein; a buffer; and a 0.5% (w/v) to 4.0% (w/v) amino acid, wherein the pH is from 5.5 to 6.5.

In addition, in another example, the liquid formulation may include: a 10 mg/mL to 90 mg/ml fusion protein; 10 mM to 50 mM histidine; and 1.0% (w/v) to 4.0% (w/v) serine, but the liquid formulation of the present invention is not limited thereto.

The liquid formulation of the present invention may include the α-galactosidase A fusion protein at a concentration of about 1 mg/mL to 150 mg/mL, about 1 mg/mL to 130 mg/mL, about 1 mg/mL to 100 mg/mL, about 5 mg/mL to 100 mg/mL, about 5 mg/mL to 90 mg/mL, about 10 mg/mL to 90 mg/mL, about 10 mg/mL to 50 mg/mL, or about 50 mg/mL to 90 mg/mL, without being limited thereto.

As used herein, the term “about” refers to a range including all of ±0.5, 0.4, ±0.3, ±0.2, ±0.1, ±0.01 or the like and includes all numerical values equivalent to those which come immediately after the term “about” or those in a similar range, without being limited thereto.

It is generally known that as a concentration of a protein contained in a formulation increases, it is difficult to maintain stability of the formulation and a problem of precipitates occurs. However, since the liquid formulation of the present invention has a composition capable of maintaining the structure of the fusion protein without causing precipitates although the fusion protein is contained at a high concentration, the α-galactosidase A fusion protein may be contained therein, as an active ingredient, for example, at a concentration of about 10 mg/mL or more, about 15 mg/ml or more, about 35 mg/ml or more, about 50 mg/mL, or about 90 mg/mL.

The buffer, as a component contained in the liquid formulation of the present invention, is a solution used to maintain the pH of the liquid formulation to stabilize the α-galactosidase A fusion protein. The liquid formulation of the present invention may include the buffer as a solvent of the liquid formulation, and any buffer may be used without limitation as long as the buffer maintains the pH level in which the α-galactosidase A fusion protein, as a target substance of stabilization, is stabilized.

In an experiment of the present invention, it was confirmed that the structure of the fusion protein is changed by the pH level of the liquid formulation, specifically, the highest structural stability of the fusion protein was obtained at a pH of 5.5 to 6.5. In addition, among the components constituting the fusion protein, the α-galactosidase A is more significantly affected by the pH level than the immunoglobulin Fc region, to be unfolded in accordance with the pH value. Therefore, it is important to maintain the structural stability of the α-galactosidase A fusion protein by maintaining the pH of the liquid formulation at an appropriate level.

The buffer may include phosphoric acid and an alkali salt thereof, as a conjugate salt (e.g., phosphate: sodium phosphate, potassium phosphate, or hydrogen or dihydrogen salt thereof), citric acid and a salt thereof (e.g., sodium citrate), acetic acid and a salt thereof (e.g., sodium acetate), or histidine and a salt thereof, and any mixture thereof may be used as the buffer, without being limited thereto. For example, the buffer may be selected from a citric acid buffer (e.g., sodium citrate buffer), an acetic acid buffer (e.g., sodium acetate buffer), a phosphoric acid buffer (e.g., sodium phosphate buffer), a histidine buffer, and any combination thereof, and the buffer or the substance used as a buffer in the liquid formulation (citric acid and a salt thereof, acetic acid and a salt thereof, histidine and a salt thereof, phosphoric acid and a salt thereof, or any combination thereof) may be contained at a concentration sufficient to maintain a target pH of the liquid formulation.

The pH of the liquid formulation may be from about 5.0 to 8.0, for example, from about 5.0 to 7.5, from about 5.0 to 7.0, from about 5.0 to 6.5, from about 5.0 to 6.0, from about 5.5 to 8.0, from about 5.5 to 7.5, from about 5.5 to 7.0, from about 5.5 to 6.5, from about 5.5 to 6.3, from about 5.5 to 6.2, from about 5.5 to 6.1, from about 5.5 to 6.0, from about 5.5 to 5.9, from about 5.5 to 5.8, from about 5.5 to 5.7, from about 5.5 to 5.6, from about 6.0 to 6.5, or about 5.0, 5.1, 5.2, 5.3, 5.4, 5.5, 5.6, 5.7, 5.8, 5.9, 6.0, 6.1, 6.2, 6.3, 6.4, or 6.5, but is not particularly limited thereto.

In order to obtain the target pH, the buffer may include phosphoric acid, citric acid, acetic acid, histidine, or salts or mixture thereof at a concentration of about 1 mM to about 200 mM, about 5 mM to about 100 mM, about 5 mM to about 80 mM, about 5 mM to about 40 mM, about 8 mM to about 40 mM, about 5 mM to about 30 mM, or about 5 mM to about 25 mM, about 10 mM to about 50 mM, about 10 mM to about 25 mM, about 15 mM to about 25 mM, or about 20 mM, without being limited thereto.

In a specific embodiment, the buffer may include 20 mM histidine and have a pH of about 5.5 to 6.5, without being limited thereto.

The amino acid, as another type of stabilizer contained in the liquid formulation of the present invention, may maintain the state of the soluble aggregates of the α-galactosidase A fusion protein in the liquid formulation. Once insoluble aggregates are formed as the structural deformation (unfolding) further proceeds from the soluble aggregates, problems such as precipitation and loss of enzymatic activity occur.

In the Experimental Example of the present invention, it was confirmed that the progress of unfolding of the α-galactosidase A fusion protein is inhibited by adding the amino acid, so that formation of higher-order aggregates is prevented and formation of insoluble aggregates therefrom is prevented. Also, it was confirmed that the amino acid prevents precipitates of the protein by inhibiting formation of higher-order aggregates and has a great influence on the increase in stability in a liquid formulation having a high concentration of the α-galactosidase A fusion protein. Because formation of the soluble aggregates or higher-order aggregates may cause immunogenicity when administered to the body, it is required to inhibit formation of the soluble aggregates or higher-order aggregates.

The amino acid contained in the liquid formulation of the present invention may be arginine, lysine, serine, threonine, asparagine, glutamine, glycine, proline, alanine, valine, isoleucine, leucine, phenylalanine, or any combination thereof, specifically, may be selected from the group consisting of arginine, serine, threonine, glutamine, glycine, alanine, and any combination thereof, without being limited thereto. As a specific example, the amino acid may be serine, without being limited thereto.

In the liquid formulation of the present invention, the amino acid may be present in an amount of about 0.1% (w/v) to 5.0% (w/v), about 0.5% (w/v) to 5.0% (w/v), about 0.5% (w/v) to 4.0% (w/v), about 1.0% (w/v) to 5.0% (w/v), about 1.0% (w/v) to 4.0% (w/v), about 2.0% (w/v) to 5.0% (w/v), about 2.5% (w/v) to 5% (w/v), about 3.0% (w/v) to 5% (w/v), about 3.5% (w/v) to 5% (w/v), about 2.0% (w/v) to 4.5% (w/v), about 2.0% (w/v) to 4.0% (w/v), or about 1.0% (w/v), about 2.0% (w/v), about 3.0% (w/v), about 4.0% (w/v), or about 5.0% (w/v), without being limited thereto.

The liquid formulation according to the present invention may further include an isotonic agent. The isotonic agent is a substance added to adjust an osmotic pressure of the liquid formulation, and any isotonic agent may be contained in the liquid formulation of the present invention without limitation as long as the isotonic agent contributes to stabilization of the α-galactosidase A fusion protein while maintaining an appropriate osmotic pressure.

The isotonic agent may include a water-soluble inorganic salt, specifically, the liquid formulation of the present invention may include sodium chloride as an isotonic agent, without being limited thereto.

In the Experimental Example of the present invention, it was confirmed that the isotonic agent inhibits formation of soluble aggregates and helps the native form of the fusion protein to be maintained in the liquid formulation of the present invention.

The concentration of sodium chloride used in the present invention may be from about 0 mM to 300 mM, specifically, from about 0.1 mM to 300 mM, may also be from about 5 mM to 300 mM, from about 5 mM to 200 mM, from about 5 mM to 150 mM, from about 5 mM to 100 mM, from about 10 mM to 200 mM, from about 10 mM to 150 mM, from about 10 mM to 100 mM, from about 30 mM to 200 mM, from about 30 mM to 150 mM, from about 30 mM to 100 mM, from about 50 mM to 200 mM, from about 50 mM to 150 mM, or from about 50 mM to 100 mM, but is not limited thereto, and may be appropriately adjusted such that formulations including all mixtures should be isotonic in accordance with types and amounts of components contained in the formulations.

The liquid formulation according to the present invention may further include a non-ionic surfactant. The non-ionic surfactant lowers surface tension of a protein solution to prevent the protein from being adsorbed to or aggregating on a hydrophobic surface.

Examples of the non-ionic surfactant available in the present invention may include polysorbates (e.g., polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), polysorbate 80 (polyoxyethylene (20) sorbitan monooleate); wherein the number 20 following the polyoxyethylene indicates a total number of oxyethylene groups (—(CH2CH2O)—)), poloxamer (PEO-PPO-PEO copolymer; wherein PEO: poly(ethylene oxide) and PPO: poly(propylene oxide)), polyethylene-polypropylene glycol, a polyoxyethylene compound (e.g., polyoxyethylene-stearate, polyoxyethylene alkyl ether (alkyl: C1-C30), polyoxyethylene monoallyl ether, alkylphenyl polyoxyethylene copolymer (alkyl: C1-C30), and the like), and sodium dodecyl sulphate (SDS), or may be polysorbate or poloxamer, used alone or in a combination of at least two thereof.

Specifically, the non-ionic surfactant may be poloxamer 188, polysorbate 20, polysorbate 40, polysorbate 60, or polysorbate 80, which may be used in combination, without being limited thereto.

In the present invention, the non-ionic surfactant may be contained in the liquid formulation of the present invention at a concentration of about 0.2% (w/v) or less, for example, about 0.001% (w/v) to about 0.2% (w/v), about 0.001% (w/v) to about 0.1% (w/v), about 0.005% (w/v) to about 0.1% (w/v), about 0.001% (w/v) to about 0.08% (w/v), about 0.002% (w/v) to about 0.08% (w/v), about 0.005% (w/v) to about 0.05% (w/v), about 0.01% (w/v) to about 0.05% (w/v), or about 0.05% (w/v), without being limited thereto.

The liquid formulation according to the present invention may further include a sugar. The sugar refers to a monosaccharide, disaccharide, polysaccharide, and oligosaccharide and may increase stability of the α-galactosidase A fusion protein in the liquid formulation. Examples thereof may include mannose, glucose, fructose, galactose, fucose, lactose, maltose, sucrose, trehalose, raffinose, dextran, or any combination thereof, and in a specific embodiment, the sugar may be glucose, fructose, galactose, lactose, maltose, sucrose, trehalose, or any combination thereof, without being limited thereto. For example, the sugar may be sucrose, without being limited thereto.

The aqueous solution according to the present invention may further include a sugar alcohol, and the sugar alcohol refers to a substance including multiple hydroxyl group and includes a substance in which aldehyde groups and/or ketone groups of sugar are substituted with alcohol groups and a sugar containing multiple hydroxyl groups. The sugar or sugar alcohol may increase stability of the α-galactosidase A fusion protein. For example, the sugar alcohol may include at least one selected from the group consisting of mannitol and sorbitol, without being limited thereto.

The sugar alcohol, sugar, or any combination thereof may be present in the liquid formulation at a concentration of about 0.5% (w/v) to 20% (w/v), about 0.5% (w/v) to 15% (w/v), about 0.5% (w/v) to 10% (w/v), about 0.5% (w/v) to 8% (w/v), about 0.5% (w/v) to 5% (w/v), about 0.5% (w/v) to 4% (w/v), about 1% (w/v) to 20% (w/v), about 1% (w/v) to 15% (w/v), about 1% (w/v) to 10% (w/v), about 1% (w/v) to 8% (w/v), about 1% (w/v) to 6% (w/v), about 1% (w/v) to 5% (w/v), or about 0.0% (w/v), about 1.0% (w/v), about 3.0% (w/v), about 4.0% (w/v), about 5.0% (w/v), or about 8.0% (w/v) based on the entire solution of the liquid formulation, without being limited thereto.

The liquid formulation according to the present invention may further include a preservative. The preservative, as a substance that substantially reduces the action of bacteria and fungi in a formulation, is added to the formulation for easy production of the formulation for multiple administration. Examples of potential preservative may include octadecyldimethyl-benzyl ammonium chloride, hexamethonium chloride, benzalkonium chloride (a mixture of alkylbenzyldimethylammonium chloride wherein an alkyl group is a long-chain compound), and benzethonium chloride. Other types of preservative may be aromatic alcohol such as phenol, butyl, or benzyl alcohol; alkyl paraben such as methyl or propyl paraben; catechol, resorcinol, cyclohexanol, 3-pentanol, and m-cresol, without being limited thereto. The concentration of the preservative may be from 0.001% (w/v) to 1.0% (w/v), without being limited thereto.

Meanwhile, the liquid formulation of the present invention may further optionally include other components or substances known in the art in addition to the above-described buffer, isotonic agent, amino acid, non-ionic surfactant, and preservative within a range that does not impair the effect of the present invention, without being limited thereto.

As a specific example, the liquid formulation according to the present invention may be a liquid formulation including a 10 mg/mL to 90 mg/mL fusion protein; 10 mM to 50 mM histidine; and 1.0% (w/v) to 4.0% (w/v) serine, without being limited thereto.

As another specific example, the liquid formulation according to the present invention may be a liquid formulation including a 10 mg/mL to 90 mg/mL fusion protein; 10 mM to 30 mM histidine; and 2.0% (w/v) to 4.0% (w/v) serine, wherein the pH is from 5.5 to 6.0, without being limited thereto.

As another specific example, the liquid formulation according to the present invention may be a liquid formulation including a 10 mg/mL to 90 mg/mL fusion protein; 10 mM to 30 mM histidine; 50 mM to 150 mM sodium chloride; and 2.0% (w/v) to 4.0% (w/v) serine, wherein the pH is from 5.5 to 6.0, without being limited thereto.

In addition, the liquid formulation according to the present invention may further include a pharmaceutically acceptable carrier, an excipient, and the like, and these carrier and excipient may be non-naturally occurring.

Meanwhile, hereinafter, the α-galactosidase A fusion protein contained in the liquid formulation of the present invention, as an active ingredient, will be described in more detail.

The α-galactosidase A fusion protein contained in the liquid formulation of the present invention refers to a fusion protein in which α-galactosidase A is linked to an immunoglobulin Fc region, and the fusion protein may have a structure in which two molecules of α-galactosidase A are linked to the immunoglobulin Fc region in a dimeric form via a linker. Specifically, two α-galactosidase A molecules may be linked to each monomer of the immunoglobulin Fc region in a dimeric form via a linker. In addition, the two α-galactosidase A molecules may form a dimer via a non-covalent bond, without being limited thereto. The fusion protein of the present invention has increased stability of α-galactosidase A since the immunoglobulin Fc region is fused to the α-galactosidase A and thus the pharmacological efficacy may be maintained for a long time in the body. The liquid formulation of the present invention does not lose the pharmacological efficacy even after long-term storage because the liquid formulation further includes a stabilizer and high stability is obtained thereby.

Specifically, the fusion protein of the present invention may have an amino acid sequence of SEQ ID NO: 4, or may be encoded by a polynucleotide having a nucleotide sequence of SEQ ID NO: 5, without being limited thereto. In the fusion protein of the present invention, two monomers including the amino acid sequence of SEQ ID NO: 4 may form a dimer, without being limited thereto. For the fusion protein of the present invention, the specification of WO 2019/009684 is incorporated as a reference.

As used herein, the terms “fusion protein in which α-galactosidase A and an immunoglobulin Fc region are fused”, “α-galactosidase A fusion protein”, and “fusion protein” may be interchangeably used.

The fusion protein of the present invention is expressed in a transformant in a form where α-galactosidase A is linked to the immunoglobulin Fc region via a linker such that α-galactosidase A forms a dimer via a non-covalent bond when the immunoglobulin Fc region forms a dimer.

The α-galactosidase A (α-Gal A, GLA) of the present invention, which is an enzyme present in lysosomes of spleen, brain, liver, and the like and hydrolyzes α-galactosyl moieties at ends of glycolipids and glycoproteins, is a homodimeric glycoprotein. Particularly, α-galactosidase A is known to be associated with Fabry disease that is a lysosomal storage disease. The α-galactosidase A has a dimeric structure consisting of two domains (TIM barrel domain and β-sheet containing immunoglobulin-like domain) (Journal of Biological chemistry, Vol. 287, No. 34, 2012; Lieberman et al. Biochemistry, Vol. 48, No. 22, 2009), and an unfolding phenomenon occurs more easily as the pH increases, and therefore the pH of a liquid formulation containing α-galactosidase A is required to be appropriately adjusted. Particularly, while the active site is maintained in the state of soluble aggregates formed as one domain is unfolded, the activity is lost once insoluble aggregates are formed as unfolding further proceeds, and thus the liquid formulation is required to include a stabilizer to keep the enzyme in the active state by maintaining the soluble aggregate state. Furthermore, since the soluble aggregates increase the risk of immunogenicity, there is a high risk of inducing immune response when the liquid formulation is administered to the body. Therefore, it is important to increase medication safety by increasing stability of a formulation to prevent formation of soluble aggregates as much as possible during storage of the formulation.

In the present invention, the α-galactosidase A may be a native form or a recombinant form, specifically, include an amino acid sequence of SEQ ID NO: 1, without being limited thereto.

In addition, the α-galactosidase A of the present invention includes fragments of the native form or analogs thereof in which one or several amino acids are altered by one selected from the group consisting of substitution, addition, deletion, modification, and any combination thereof, without limitation, as long as they have activity identical or equivalent to that of the native form of the enzyme.

Additionally, the α-galactosidase A may include an amino acid sequence having at least 60%, 70%, or 80%, specifically at least 90%, more specifically, at least 91%, 92%, 93%, 94%, 95%, 96%, 97%, 98%, or 99% homology with the amino acid sequence of SEQ ID NO: 1 and may be obtained from microorganisms by using a genetic engineering method or be commercially available, without being limited thereto.

As used herein, the term “homology” refers to a degree of identity with a wild-type amino acid sequence or a wild-type nucleotide sequence, and the homology comparison may be done by visual observation or using a commercially available comparison program. Using a commercially available computer program, the homology between two or more sequences may be expressed as a percentage (%). The homology (%) may be calculated between adjacent sequences.

Genetic information of sequences of the α-galactosidase A or derivatives thereof and nucleotide sequences encoding the same may be available from known database such as the National Center for Biotechnology Information (NCBI).

The α-galactosidase A fusion protein of the present invention may be prepared by expressing the fusion protein in a transformant in a form where α-galactosidase A is linked to the immunoglobulin Fc region via a linker.

The linker is a peptide linker, and the fusion protein of the present invention may be in a form in which the α-galactosidase A is linked to the immunoglobulin Fc region via the peptide linker. One end of the linker may be linked to one of the chains of the immunoglobulin Fc region in a dimer form, without being limited thereto.

The peptide linker may include one or more amino acids, e.g., 1 to 1000 amino acids, and include any peptide linker known in the art, such as [GS]x linker, [GGGS]x linker, and [GGGGS]x linker, wherein x is a natural number of 1 or greater (e.g., 1, 2, 3, 4, 5, or more), without being limited thereto. Specifically, the peptide linker of the present invention may consist of 10 to 50 amino acids, more specifically, 20 to 40 amino acids and may have an amino acid sequence of SEQ ID NO: 2.

In view of the objects of the present invention, sites of the α-galactosidase A and the immunoglobulin Fc region to which the peptide linker is linked are not particularly limited as long as the activity of the α-galactosidase A is retained after being linked to the immunoglobulin Fc region. Specifically, the sites may be both termini of the α-galactosidase A and the immunoglobulin Fc region, more specifically, the C-terminus of the α-galactosidase A and the N-terminus of the immunoglobulin Fc region, without being limited thereto.

As used herein, the term “N-terminus” or “C-terminus” refers to the amino terminus and the carboxyl terminus of the respective proteins. Examples thereof include, but are not limited to, not only the last amino acid residues of the N-terminus or the C-terminus but also amino acid residues near the N-terminus or the C-terminus, specifically, up to the 10th amino acid residues from the last amino acid.

In the present invention, the peptide linkers may be linked to each of the monomers of the immunoglobulin Fc region in a dimeric form, and the linkers respectively linked to the immunoglobulin Fc region monomers of the dimer may be independently linked to the α-galactosidase A, without being limited thereto.

The immunoglobulin Fc region, one of the moieties constituting the enzyme fusion protein of the present invention, may be a dimer formed of immunoglobulin Fc region monomers.

As used herein, the term “immunoglobulin Fc region” refers to a region including a heavy chain constant domain 2 (CH2) and/or a heavy chain constant domain 3 (CH3) excluding the heavy chain and light chain variable domains of immunoglobulin. In view of the objects of the present invention, the immunoglobulin Fc region may include a modified hinge region, without being limited thereto. Specifically, the immunoglobulin Fc region may be one in which one or more amino acids are altered from the native form of the immunoglobulin Fc region by one selected from the group consisting of substitution, addition, deletion, modification, and any combination thereof, without being limited thereto.

The immunoglobulin Fc region is a substance used as a carrier in drug production. In order to stabilize a protein and prevent the protein from being eliminated by the kidney, extensive research has been conducted into fusion proteins using the immunoglobulin Fc region in recent years. Immunoglobulins are major constituents of the blood, and there are five different types, i.e., IgG, IgM, IgA, IgD, and IgE. The most frequently used type for fusion protein studies is IgG, and it is classified into four subtypes (IgG1 to IgG4).

The immunoglobulin Fc region may include a hinge region in the heavy chain constant domain, the immunoglobulin Fc region monomers may constitute a dimer via the hinge region, without being limited thereto. In addition, the immunoglobulin Fc region of the present invention may be an extended Fc region including a part of or the entirety of a heavy chain constant domain 1 (CH1) and/or a light chain constant domain 1 (CL1) excluding the heavy chain and the light chain variable domains of the immunoglobulin, as long as the immunoglobulin Fc region has substantially identical or enhanced effects compared to the native form. In addition, the immunoglobulin Fc region may be a region from which a considerably long part of the amino acid sequence corresponding to the CH2 and/or CH3 is eliminated.

Specifically, the immunoglobulin Fc region of the present invention may include 1) a CH1 domain, a CH2 domain, a CH3 domain, and a CH4 domain, 2) a CH1 domain and a CH2 domain, 3) a CH1 domain and a CH3 domain, 4) a CH2 domain and a CH3 domain, or 5) a combination of one or more domains selected from a CH1 domain, a CH2 domain, a CH3 domain, and a CH4 domain and an immunoglobulin hinge region (or a part of the hinge region), but is not limited thereto. More specifically, the immunoglobulin Fc region may include a hinge region, a CH2 domain, and a CH3 domain, but is not limited thereto.

As used herein, the term “hinge sequence” refers to a site located at a heavy chain and forming the dimer of the immunoglobulin Fc region via an inter disulfate bond. In the present invention, the dimer is formed of two molecules of the immunoglobulin Fc chain via the hinge sequence of the immunoglobulin Fc region.

Specifically, the hinge region may be one where a part of the hinge region is deleted to have only one cysteine (Cys) residue, or one where a serine (Ser) residue involved in chain exchange is substituted with a proline (Pro) residue. More specifically, the hinge region may be one where the 2^{nd} serine residue is substituted with a proline residue, without being limited thereto. The immunoglobulin Fc region of the present invention may have an amino acid sequence of SEQ ID NO: 3, without being limited thereto.

The immunoglobulin Fc region of the present invention include not only a native sequence obtained from papain digestion of immunoglobulin but also derivatives, substitution products, and variants thereof, e.g., sequences different from the native sequence and obtained by modification of one or more amino acid residues by deletion, addition, non-conservative or conservative substitution, or a combination thereof. The derivatives, substitution products, and variants are those having the ability to binding FcRn.

For example, in the case of IgG Fc, amino acid residues known to be important in linkage at positions 214 to 238, 297 to 299, 318 to 322, or 327 to 331 may be used as a suitable site for modification.

Additionally, other various types of derivatives including those in which a site capable of forming a disulfide bond is deleted or certain amino acid residues are eliminated from the N-terminus of a native Fc form, and a methionine residue is added to the N-terminus of the native Fc form may be used. In addition, to remove effector functions, a complement-binding site, such as a C1q-binding site, may be deleted, and an antibody dependent cell mediated cytotoxicity (ADCC) site may be deleted. Techniques of preparing such sequence derivatives of the immunoglobulin Fc region are disclosed in International Patent Publication Nos. WO 97/34631 and WO 96/32478.

Amino acid exchanges in proteins and peptides, which do not generally alter the activity of molecules, are known in the art (H. Neurath, R. L. Hill, The Proteins, Academic Press, New York, 1979). The most commonly occurring exchanges of amino acid residues are exchanges between Ala/Ser, Val/Ile, Asp/Glu, Thr/Ser, Ala/Gly, Ala/Thr, Ser/Asn, Ala/Val, Ser/Gly, Thy/Phe, Ala/Pro, Lys/Arg, Asp/Asn, Leu/Ile, Leu/Val, Ala/Glu, and Asp/Gly. If required, the Fc region may be modified by phosphorylation, sulfation, acrylation, glycosylation, methylation, farnesylation, acetylation, and amidation.

The above-described sequence derivatives of the immunoglobulin Fc region are derivatives that have biological activity equivalent to the immunoglobulin Fc region of the present invention and improved structural stability against heat, pH, or the like.

In addition, these immunoglobulin Fc regions may be obtained from native forms isolated from humans and other animals including cows, goats, swine, mice, rabbits, hamsters, rats and guinea pigs, or may be recombinants or derivatives thereof, obtained from transformed animal cells or microorganisms. In this regard, they may be obtained from a native immunoglobulin by isolating whole immunoglobulins from living humans or animals and treating them with a protease. Papain digests the native immunoglobulin into Fab and Fc regions and pepsin digests the native immunoglobulin into pF′c and F(ab)2 fragments. These fragments may be subjected to size-exclusion chromatography to isolate Fc or pF′c. In a more specific embodiment, a human-derived immunoglobulin Fc region is a recombinant immunoglobulin Fc region obtained from a microorganism.

In addition, the immunoglobulin Fc region may have natural glycans or increased or decreased glycans compared to the natural type, or be in a deglycosylated form. The increase, decrease, or removal of glycans of the immunoglobulin Fc may be achieved by any methods commonly used in the art such as a chemical method, an enzymatic method, and a genetic engineering method using a microorganism. In this regard, the immunoglobulin Fc region obtained by removing glycans shows a significant decrease in binding affinity to a complement c1q and a decrease in or loss of antibody-dependent cytotoxicity or complement-dependent cytotoxicity, and thus unnecessary immune responses are not induced thereby in living organisms. Based thereon, a deglycosylated or aglycosylated immunoglobulin Fc region may be more suitable as a drug carrier for its own purpose.

As used herein, the term “deglycosylation” refers to a Fc region from which glycan is removed using an enzyme and the term “aglycosylation” refers to a Fc region that is not glycosylated and produced in prokaryotes, more specifically, E. coli.

Meanwhile, the immunoglobulin Fc region may be derived from humans or animals such as cows, goats, swine, mice, rabbits, hamsters, rats, or guinea pigs. In a more specific embodiment, the immunoglobulin Fc region may be derived from humans.

In addition, the immunoglobulin Fc region may be derived from IgG, IgA, IgD, IgE, or IgM, or any combination or hybrid thereof. In a more specific embodiment, the immunoglobulin Fc region is derived from IgG or IgM which are the most abundant proteins in human blood, and in an even more specific embodiment, it is derived from IgG known to enhance the half-lives of ligand-binding proteins. In a yet even more specific embodiment, the immunoglobulin Fc region is an IgG4 Fc region, and in the most specific embodiment, the immunoglobulin Fc region is an aglycosylated Fc region derived from human IgG4, without being limited thereto. The immunoglobulin Fc region of the present invention may have an amino acid sequence of SEQ ID NO: 3, but is not limited thereto. More specifically, the immunoglobulin Fc region may include a monomer having an amino acid sequence of SEQ ID NO: 3, and the immunoglobulin Fc region may be a homodimer of the monomers having an amino acid sequence of SEQ ID NO: 3, without being limited thereto.

Meanwhile, as used herein, the term “combination” refers to formation of a linkage between a polypeptide encoding a single-chain immunoglobulin Fc region of the same origin and a single-chain polypeptide of a different origin when a dimer or a multimer is formed. That is, a dimer or multimer may be prepared using two or more Fc fragments selected from the group consisting of IgG Fc, IgA Fc, IgM Fc, IgD Fc, and IgE Fc fragments.

The immunoglobulin Fc region of the α-galactosidase A fusion protein may be in a dimeric form, specifically, may have a structure in which two polypeptide chains are linked to each other via a disulfide bond. More specifically, two chains may be linked via the nitrogen atom of one of the two chains, without being limited thereto. The linkage via the nitrogen atom may be formed by reductive amination of an ε-amino group of lysine or an N-terminal amino group, without being limited thereto. In a specific embodiment, the immunoglobulin Fc region may be linked to the linker via a nitrogen atom of proline at the N-terminus, without being limited thereto. One Fc region of the dimeric form may be linked to two α-galactosidase A molecules by covalent bonds via two linkers, without being limited thereto.

Unless otherwise stated, detailed descriptions about the α-galactosidase A or fusion protein of the present invention disclosed in the specification or the claims may be applied not only to the α-galactosidase A or fusion protein but also to a salt thereof (e.g., pharmaceutically acceptable salt), or a solvate form thereof. Thus, although only “α-galactosidase A” or “fusion protein” are described in the specification, descriptions thereof may also be applied to particular salts thereof, particular solvates thereof, and solvates of the particular salts. Such salts may be, for example, in the form of a pharmaceutically acceptable salt. Types of the salts are not particularly limited. However, the salts may be in a form safe and effective in mammals, without being limited thereto.

The term “pharmaceutically acceptable” refers to a substance that may be effectively used for the intended use within the scope of pharmaco-medical decision without inducing excessive toxicity, irritation, allergic responses, and the like.

As used herein, the term “pharmaceutically acceptable salt” refers to a salt derived from a pharmaceutically acceptable inorganic acid, organic acid, or base. Examples of a suitable acid may include hydrochloric acid, bromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, citric acid, methanesulfonic acid, formic acid, benzoic acid, malonic acid, naphthalene-2-sulfonic acid, and benzenesulfonic acid. Examples of the salt derived from a suitable base may include alkali metals such as sodium and potassium, alkali earth metals such as magnesium, and ammonium.

In addition, as used herein, the term “solvate” refers to a complex of the α-galactosidase A, fusion protein or the salt thereof according to the present invention and a solvent molecule.

The fusion protein may be prepared or produced by any method known in the art, specifically, may be obtained from animal cells into which an expression vector is inserted after culturing and purifying the animal cells, or may be synthesized based on the sequence thereof, without being limited thereto.

The liquid formulation of the present invention may be used to prevent, treat, and alleviate a disease, such as α-galactosidase A deficiency, whose symptoms may be prevented, treated, or alleviated by administering the α-galactosidase A fusion protein, without being limited thereto.

The α-galactosidase A fusion protein of the present invention may be used as a drug of an enzyme replacement therapy (ERT). The enzyme replacement therapy may prevent or treat a disease by recovering hypofunction of an enzyme by supplementing the deficient or insufficient enzyme that causes the disease.

Specifically, the liquid formulation of the present invention may be used to prevent, treat, or alleviate α-galactosidase A deficiency. The α-galactosidase A deficiency is a lysosomal storage disease caused by deficiency of α-galactosidase A (α-Gal A) that is a lysosomal enzyme and include Fabry disease, Angiokeratoma Diffuse, Angiokeratoma Corporis Diffusum, or Hereditary Dystopic Lipidosis.

Fabry disease, one of the lysosomal storage diseases, is a recessively inherited disorder caused by X-chromosomal inactivation. Fabry disease is a congenital metabolic disorder of glycolipid (glycosphingolipid) caused by deficient or insufficient activity of α-galactosidase A. It has been known that abnormal accumulation of globotriaosylceramide (Gb3) on the blood vessel wall and various parts of the body, such as skin, kidneys, heart, and nervous system, caused by the abnormality of α-galactosidase A significantly affects blood circulation and supply of nutrients. Symptoms such as hypohidrosis, severe pain, acroparesthesia, angiokeratoma, corneal opacity, cardiac ischemia, myocardial infarction, and renal failure are caused, and eventually the kidneys failed to function properly, leading to death.

As used herein, the term “prevention” refers to all actions that inhibit a disease by administering the fusion protein or a liquid formulation including the same or that inhibit or delay the onset of the disease by administering the liquid formulation. As used herein, the term “treatment” refers to all actions that ameliorate or beneficially change symptoms of a target disease by administering the fusion protein or a liquid formulation including the same.

In a specific embodiment of the present invention, the liquid formulation of the present invention may be used to prevent or treat the Fabry disease, without being limited thereto.

In addition, the liquid formulation of the present invention may be a formulation or composition suitable for subcutaneous administration and administered via a subcutaneous route, without being limited thereto.

As used herein, the term “administration” refers to introduction of the liquid formulation into a patient by any suitable method, and administration routes of the liquid formulation are not particularly limited, but the liquid formulation may be administered by any general route as long as the α-galactosidase A fusion protein of the liquid formulation reaches a target in the living body, for example, intraperitoneal administration, intravenous administration, intramuscular administration, subcutaneous administrations, intradermal administration, oral administration, topical administration, intranasal administration, intrapulmonary administration, or rectal administration, without being limited thereto.

A preferred daily dosage of the fusion protein of the present invention may be from about 0.0001 mg to 500 mg per 1 kg of the body weight of a patient and the liquid formulation may be formulated such that the daily dosage is properly administered. However, the dosage of the fusion protein is determined as an effective dosage for the patient in consideration of various factors such as age, body weight, health status, and gender of the patient, severity of disease, diet, and excretion rate as well as administration route and number of administration thereof, and thus an appropriate effective dosage for a particular use of the liquid formulation of the present invention may be determined by one of ordinary skill in the art in consideration of these factors.

Another aspect of the present invention provides a method for preparing the liquid formulation.

Specifically, the preparation method may include mixing an α-galactosidase A fusion protein, a buffer, and an amino acid.

The liquid formulation may further include at least one selected from an isotonic agent and a non-ionic surfactant, without being limited thereto.

The liquid formulation and components constituting the liquid formulation are as described above.

Hereinafter, the present invention will be described in more detail with reference to the following examples. However, the following examples and experimental examples are merely presented to exemplify the present invention, and the scope of the present invention is not limited thereto.

### Example 1. Size-Exclusion Liquid Chromatography (SE-HPLC)

For size-exclusion chromatography, first, a sample was diluted using a mobile phase (1×PBS, Lonza) to a concentration of 1.0 mg/mL, followed by sterile filtration, and 200 μL of the filtered sample was injected into a vial insert and prepared in a screw-top vial.

Subsequently, after connecting the mobile phase to a pump, an assay column (TSKgel G3000SWXL, Tosoh) was loaded on Waters e2695 and Waters 2489 devices (manufactured by Waters, Japan) while flowing the mobile phase at a flow rate of 0.5 mL/min. The sample was equilibrated by flowing the mobile phase at a flow rate of 0.5 mL/min for 30 minutes or more until detector signals were stabilized. When the temperature of an autosampler was lowered to 4° C., the sample was inserted into the sampler. After injecting 10 μL of the sample, detection peaks were identified at 214 nm while flowing the mobile phase for 35 minutes.

The results were analyzed using Empower Pro software of a personal computer (PC).

### Example 2. Thermal Unfolding and Thermal Aggregation Assay

The degree of protein unfolding may be measured by detecting emission wavelengths of tryptophan exposed on the surface when the protein was unfolded as temperature increases. Differential scanning fluorimetry based on such intrinsic fluorescence intensity was conducted using a UNcle device (Unchained Labs). In order to analyze thermal stability of the protein using the device, thermal unfolding (Tm) and thermal aggregation (Tagg) were measured as follows.

8.8 μL of the sample was injected in duplicate into a Uni sample loader (Unchained Labs), and temperature was raised from 25° C. to 95° C. at a rate of 1° C./min. While raising the temperature, intensity of wavelengths (250 nm to 720 nm) emitted at an excitation wavelengths of 266 nm was measured. Fluorescence data analysis was conducted using UNcle Analysis software (Unchained Labs).

Based on the analysis, a temperature where a fluorescent emission peak was maximum was defined as Tm, and a static light scattering of the protein at 473 nm (SLS473) was measured and a protein aggregation onset temperature, where aggregation of the protein was initiated, was defined as Tagg.

### Example 3. Biological Activity (pNP-Gal Activity) Assay

In activity assay of α-galactosidase A (α-Gal A), 4-nitrophenyl-α-D-galactopyranoside (pNP-Gal) was used as a chromogenic substrate. During hydrolysis, the pNP-Gal is degraded into galactose and 4-nitrophenol by the action of α-galactosidase A. 4-nitrophenol exhibiting a light yellow color provides a visual evidence of hydrolysis.

A buffer used for the assay is as follows.
1) Assay buffer: 20 mM citric acid, 30 mM sodium phosphate, 0.1% BSA, and 0.67% ethanol absolute (pH 4.6)
2) Substrate solution: 24.1 mg/mL pNP-Gal (Sigma) in the assay buffer
3) Stop solution: 130 mM glycine and 83 mM sodium carbonate (pH 10.6)

In vitro activity is measured as follows.

Samples diluted with the assay buffer were prepared (300 ng/ml and 150 ng/ml) and a standard solution of 4-Nitrophenol (Sigma) diluted with the assay buffer was prepared. 50 μL of the sample and the standard solution (STD) were loaded in duplicate in each well of an assay plate (Corning). The assay plate and the substrate solution were incubated at 37° C. for 30 minutes for temperature equilibrium, and then 50 μL of the substrate solution was loaded on the assay plate. After the assay plate was incubated at 37° C. for 20 minutes, 100 μL of a stop solution was loaded to stop reaction, followed by stabilization. Then, absorbance was analyzed at 405 nm (Multimode Plate Reader, PerkinElmer).

### Example 4. Turbidity Assay

Turbidity was analyzed using a Lunatic (Unchained Labs). 2.0 μL of the sample was injected into a Lunatic plate (Unchained Labs) and turbidity was measured at 350 nm. Turbidity of a placebo buffer was subtracted from turbidity of the sample.

Results of the experiment were analyzed as in the following experimental examples.

### Experimental Example 1: Preparation of Fusion Protein of α-Galactosidase A and Immunoglobulin Fc Region

A fusion protein of α-galactosidase A and immunoglobulin Fc region (hereinafter, referred to as α-galactosidase A fusion protein, SEQ ID NO: 4), in which native-type α-galactosidase A (SEQ ID NO: 1) is linked to an immunoglobulin Fc region (SEQ ID NO: 3) via a linker (SEQ ID NO: 2), was prepared.

Specifically, a polynucleotide (SEQ ID NO: 5) encoding the α-galactosidase A fusion protein was inserted into an XOGC vector, which is an expression vector, by using a restriction enzyme to prepare a vector that expresses the α-galactosidase A fusion protein.

DNA and protein sequences of the α-galactosidase A fusion protein are as shown in Table 1 below. In the protein sequences of Table 1 below, underlines indicate signal sequences, bold letters indicate amino acid substitution, and italic letters indicate the linker. The α-galactosidase A fusion protein of the present invention is in a dimeric form consisting of two monomers having an amino acid sequence of SEQ ID NO: 4.

TABLE 1

| | | SEQ ID |
|---|---|---|
| | Sequence | NO: |
| Protein | MQLRNPELHL GCALALRFLA LVSWDIPGAR ALDNGLARTP | 4 |
| | TMGWLHWERF MCNLDCQEEP DSCISEKLFM EMAELMVSEG | |
| | WKDAGYEYLC IDDCWMAPQR DSEGRLQADP QRFPHGIRQL | |
| | ANYVHSKGLK LGIYADVGNK TCAGFPGSFG YYDIDAQTFA | |
| | DWGVDLLKFD GCYCDSLENL ADGYKHMSLA LNRTGRSIVY | |
| | SCEWPLYMWP FQKPNYTEIR QYCNHWRNFA DIDDSWKSIK | |
| | SILDWTSFNQ ERIVDVAGPG GWNDPDMLVI GNFGLSWNQQ | |
| | VTQMALWAIM AAPLFMSNDL RHISPQAKAL LQDKDVIAIN | |
| | QDPLGKQGYQ LRQGDNFEVW ERPLSGLAWA VAMINRQEIG | |
| | GPRSYTIAVA SLGKGVACNP ACFITQLLPV KRKLGFYEWT | |
| | SRLRSHINPT GTVLLQLENT MQMSLKDLLG GGGSGGGGSG | |
| | GGGSGGGGSG GGGSGGGGSP PCPAPEFLGG PSVFLFPPKP | |
| | KDTLMISRTP EVTCVVVDVS QEDPEVQFNW YVDGVEVHNA | |
| | KTKPREEQFQ STYRVVSVLT VLHQDWLNGK EYKCKVSNKG | |
| | LPSSIEKTIS KAKGQPREPQ VYTLPPSQEE MTKNQVSLTC | |
| | LVKGFYPSDI AVEWESNGQP ENNYKTTPPV LDSDGSFFLY | |
| | SRLTVDKSRW QEGNVFSCSV MHEALHNHYT QKSLSLSLGK | |
| DNA | ATGCAGCTGA GGAACCCAGA ACTACATCTG GGCTGCGCGC | 5 |
| | TTGCGCTTCG CTTCCTGGCC CTCGTTTCCT GGGACATCCC | |
| | TGGGGCTAGA GCACTGGACA ATGGATTGGC AAGGACGCCT | |
| | ACCATGGGCT GGCTGCACTG GGAGCGCTTC ATGTGCAACC | |
| | TTGACTGCCA GGAAGAGCCA GATTCCTGCA TCAGTGAGAA | |
| | GCTCTTCATG GAGATGGCAG AGCTCATGGT CTCAGAAGGC | |
| | TGGAAGGATG CAGGTTATGA GTACCTCTGC ATTGATGACT | |
| | GTTGGATGGC TCCCCAAAGA GATTCAGAAG GCAGACTTCA | |
| | GGCAGACCCT CAGCGCTTTC CTCATGGGAT TCGCCAGCTA | |
| | GCTAATTATG TTCACAGCAA AGGACTGAAG CTAGGGATTT | |
| | ATGCAGATGT TGGAAATAAA ACCTGCGCAG GCTTCCCTGG | |
| | GAGTTTTGGA TACTACGACA TTGATGCCCA GACCTTTGCT | |
| | GACTGGGGAG TAGATCTGCT AAAATTTGAT GGTTGTTACT | |
| | GTGACAGTTT GGAAAATTTG GCAGATGGTT ATAAGCACAT | |
| | GTCCTTGGCC CTGAATAGGA CTGGCAGAAG CATTGTGTAC | |
| | TCCTGTGAGT GGCCTCTTTA TATGTGGCCC TTTCAAAAGC | |
| | CCAATTATAC AGAAATCCGA CAGTACTGCA ATCACTGGCG | |
| | AAATTTTGCT GACATTGATG ATTCCTGGAA AAGTATAAAG | |
| | AGTATCTTGG ACTGGACATC TTTTAACCAG GAGAGAATTG | |
| | TTGATGTTGC TGGACCAGGG GGTTGGAATG ACCCAGATAT | |
| | GTTAGTGATT GGCAACTTTG GCCTCAGCTG GAATCAGCAA | |
| | GTAACTCAGA TGGCCCTCTG GGCTATCATG GCTGCTCCTT | |
| | TATTCATGTC TAATGACCTC CGACACATCA GCCCTCAAGC | |
| | CAAAGCTCTC CTTCAGGATA AGGACGTAAT TGCCATCAAT | |
| | CAGGACCCCT TGGGCAAGCA AGGGTACCAG CTTAGACAGG | |
| | GAGACAACTT TGAAGTGTGG GAACGACCTC TCTCAGGCTT | |
| | AGCCTGGGCT GTAGCTATGA TAAACCGGCA GGAGATTGGT | |
| | GGACCTCGCT CTTATACCAT CGCAGTTGCT TCCCTGGGTA | |
| | AAGGAGTGGC CTGTAATCCT GCCTGCTTCA TCACACAGCT | |
| | CCTCCCTGTG AAAAGGAAGC TAGGGTTCTA TGAATGGACT | |
| | TCAAGGTTAA GAAGTCACAT AAATCCCACA GGCACTGTTT | |
| | TGCTTCAGCT AGAAAATACA ATGCAGATGT CATTAAAAGA | |
| | CTTACTTGGC GGCGGAGGTT CAGGTGGTGG TGGCTCTGGC | |
| | GGTGGAGGGT CGGGGGGAGG CGGCTCTGGA GGAGGGGGCT | |
| | CCGGTGGGGG AGGTAGCCCA CCATGCCCAG | |
| | CACCTGAGTTCCTGGGGGGA CCATCAGTCT TCCTGTTCCC | |
| | CCCAAAACCC AAGGACACCC TCATGATCTC CCGGACCCCT | |
| | GAGGTCACAT GCGTGGTGGT GGACGTGAGCCAGGAAGACC | |
| | CTGAGGTCCA GTTCAACTGG TACGTGGACG GCGTGGAGGT | |
| | GCATAATGCC AAGACAAAGC CGCGGGAGGA GCAGTTCCAA | |
| | AGCACGTACC GTGTGGTCAG CGTCCTCACC GTCCTGCACC | |
| | AGGACTGGCT GAATGGCAAG GAGTACAAGT GCAAGGTCTC | |
| | CAACAAAGGC CTCCCATCCT CCATCGAGAA AACCATCTCC | |
| | AAAGCCAAAG GGCAGCCCCG AGAACCACAG GTGTACACCC | |
| | TGCCCCCATC CCAGGAGGAG ATGACCAAGA ACCAGGTCAG | |
| | CCTGACCTGC CTGGTCAAAG GCTTCTATCC CAGCGACATC | |
| | GCCGTGGAGT GGGAGAGCAA TGGGCAGCCG GAGAACAACT | |
| | ACAAGACCAC GCCTCCCGTG CTGGACTCCG ACGGCTCCTT | |
| | CTTCCTCTAC AGCAGGCTAA CCGTGGACAA GAGCAGGTGG | |
| | CAGGAGGGGA ACGTCTTCTC ATGCTCCGTG ATGCATGAGG | |
| | CTCTGCACAA CCACTACACG CAGAAGAGCC TCTCCCTGTC | |
| | TCTGGGTAAA TGA | |

CHO-S cell lines were transfected with a vector (pX0GC-alpha galactosidase-Fc) that expresses the α-galactosidase A fusion protein prepared as described above to prepare cells lines capable of mass producing the α-galactosidase A fusion protein.

Specifically, CHO-S cells were suspension-cultured in a 1 L Erlenmeyer flask (Corning, cat. No. 431147) using a serum-free culture medium (FreeStyle CHO Expression Medium, Thermo Fisher, cat. No. 12651014) until the number of cells in the culture vessel reached 5×10⁸, and then the cells were transformed using a FreeStyle Max (Thermo Fisher, cat. No. 16447-100). That is, after adding 10 ml of OptiPro SFM (Thermo Fisher, cat. No. 12309-019) to each of the two tubes, and 500 μg of DNA was added to one tube and 500 μL of the FreeStyle Max was added to the other tube, followed by mixing the two solutions and the mixture was allowed to stand at room temperature of 10 minutes. Then, the culture medium was replaced with a new FreeStyle CHO expression medium (Thermo Fisher, cat. No. 12651014). The cells were incubated under the conditions of 37° C., 5% CO₂, and 125 rpm for about 96 hours to prepare the α-galactosidase A fusion protein.

In the α-galactosidase A fusion protein prepared as described above, each of the two immunoglobulin Fc region monomers constituting the dimer is linked to α-galactosidase A to form a structure in which the immunoglobulin Fc region in the dimeric form is fused to two α-galactosidase A molecules.

### Experimental Example 2: Identification of Stability of α-Galactosidase a Fusion Protein According to pH Conditions of Liquid Formulation

### Experimental Example 2-1: Preparation of Formulation

In order to identify structural changes of the α-galactosidase A fusion protein according to pH change, liquid formulations with different pH levels were prepared.

TABLE 2

| | Concentration | | | |
|---|---|---|---|---|
| | of fusion | | | |
| # | protein | Buffer | Isotonic agent | pH |
| 1 | 10 mg/mL | 20 mM L-histidine | 150 mM sodium chloride | 5.5 |
| 2 | 10 mg/mL | 20 mM L-histidine | 150 mM sodium chloride | 6.0 |
| 3 | 10 mg/mL | 20 mM L-histidine | 150 mM sodium chloride | 6.5 |
| 4 | 10 mg/mL | 20 mM L-histidine | 150 mM sodium chloride | 7.0 |
| 5 | 10 mg/mL | 20 mM L-histidine | 150 mM sodium chloride | 7.5 |
| 6 | 10 mg/mL | 20 mM L-histidine | 150 mM sodium chloride | 8.0 |

### Experimental Example 2-2: Measurement of Tm and Tagg According to pH Change

Tm and Tagg values of the formulations shown in Table 2 were measured using the UNCLE device of Example 2.

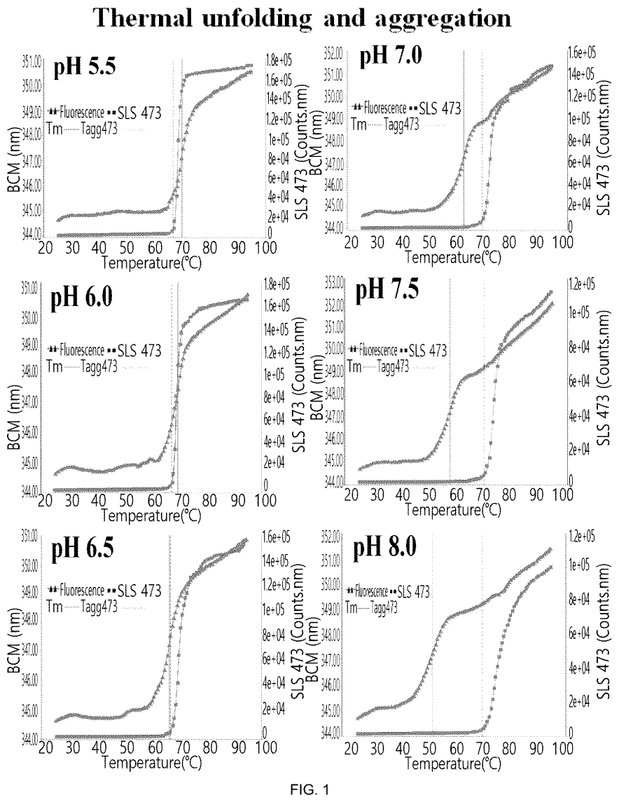

As a result, as shown in FIG. 1, it was confirmed that a liquid formulation having a lower pH (closer to pH 5.5) had a higher Tm value, and a liquid formulation having a higher pH (closer to pH 8.0) had a higher Tagg value.

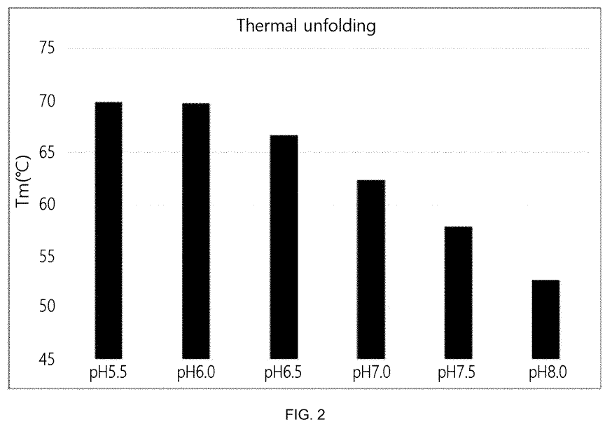

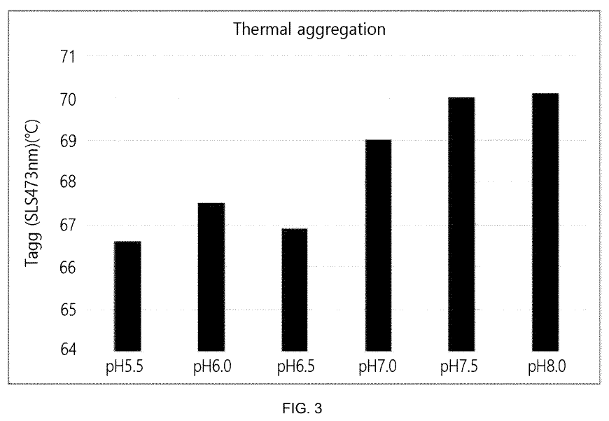

Specifically, it was confirmed that the Tm values were 69.8° C., 69.7° C., 66.6° C., 62.3° C., 57.8° C., and 52.6° C., respectively at pH 5.5, 6.0, 6.5, 7.0, 7.5, and 8.0 (FIG. 2), and the Tagg values were 66.6° C., 67.5° C., 66.9° C., 69.0° C., 70.0° C., and 70.1° C., respectively (FIG. 3).

In this case, a higher Tm value indicates a higher structural stability of a protein, and a higher Tagg value indicates a higher colloidal stability in the protein.

Based on these results, it was confirmed that the α-galactosidase A fusion protein had excellent structural stability at a low pH closer to the pH 5.5, particularly excellent at a pH of 5.5 to 6.5.

In addition, referring to FIGS. 1 to 3, it was confirmed that the variation of the Tm value according to the pH was higher than the variation of the Tagg value, and it was confirmed that the Tm value is more sensitive to pH compared to the Tagg value.

### Experimental Example 2-3: SE-HPLC Analysis According to pH Change (Room Temperature Accelerated Stability)

Stabilities of the liquid formulations having different PH levels and prepared in Experimental Example 2-1 above were compared by a room temperature accelerated stability test. Specifically, the liquid formulations of Experimental Example 2-1 were analyzed by size exclusion liquid chromatography, and all samples were analyzed at the initial point and after 7 days of storage at 25° C. (65% RH).

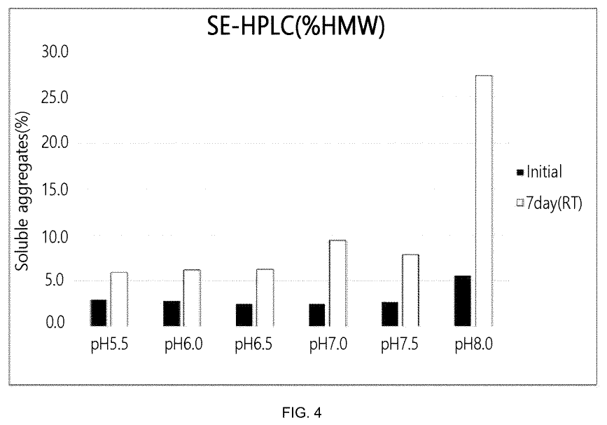

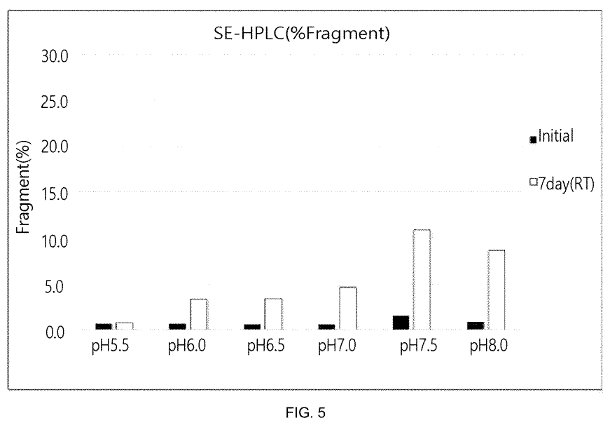

As a result, it was confirmed that formation of soluble aggregates (HMW) increased relative to the total fusion protein as the pH increases after one week of storage at room temperature (FIG. 4), and it was also confirmed that formation of the fragments increased (FIG. 5).

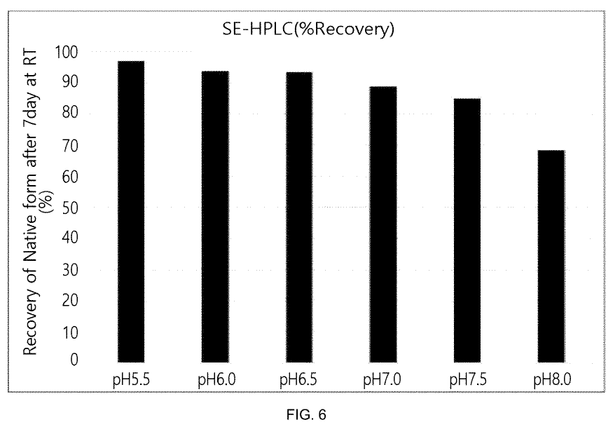

In addition, as a result of identifying recovery of native form after one week, it was confirmed that the recovery of native form decreased as the pH increases (FIG. 6).

In this regard, the soluble aggregates (HMW) are in a state where the activity of α-galactosidase A is retained while having solubility some domains of the α-galactosidase A are unfolded, the native form refers to the α-galactosidase A fusion protein prepared in Experimental Example 1 as a fusion protein in an intact dimeric form that has not undergone aggregation or degradation, and the fragments refer to a substance in which one or two molecules of α-galactosidase A are separated from the Fc region of the native form, thereby losing the structure of the α-galactosidase A fusion protein of the present invention. The definitions may be applied below in the same manner.

### Experimental Example 2-4: SE-HPLC Analysis According to pH Change (Long-Term Stability During Cold Storage, 6 Months)

Long-term stability of the liquid formulations having different pH and prepared in Experimental Example 2-1 above were identified during cold storage for 6 months. Specifically, the liquid formulations of Experimental Example 2-1 were analyzed by size exclusion liquid chromatography, and all samples were analyzed at the initial point and after 6 months of storage at 5° C.

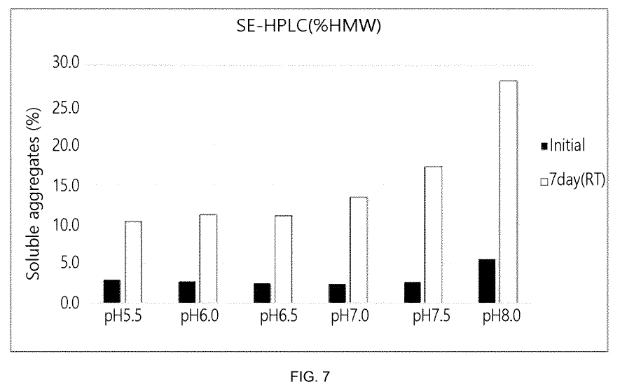

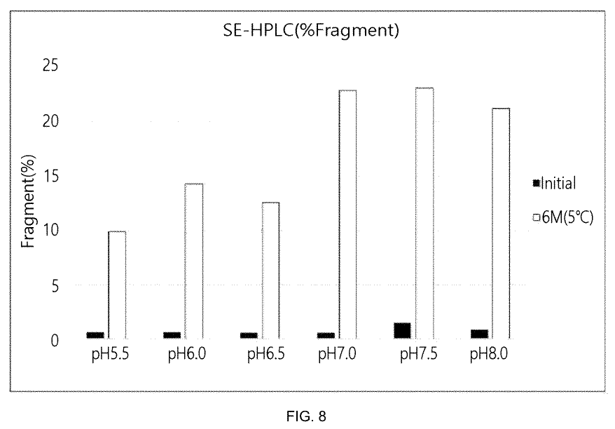

As a result, after 6 months of cold storage, it was confirmed that formation of soluble aggregates (HMW) increased as pH increased (FIG. 7), and it was also confirmed that formation of fragments increased (FIG. 8).

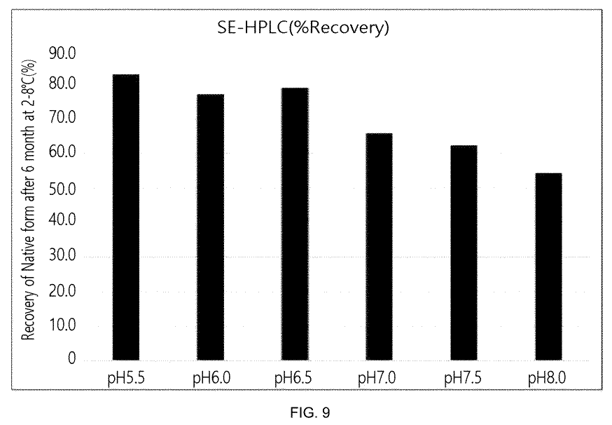

In addition, as a result of identifying recovery of native form after 6 months of cold storage, it was confirmed that the recovery of native form decreased as the pH increased (FIG. 9).

Referring to Experimental Examples 2-3 and 2-4, it was confirmed that formation of the soluble aggregates and unfolding of the α-galactosidase A fusion protein increased in the liquid formulation as the pH increased, and it was also confirmed that it is difficult to recover the structure of the α-galactosidase A fusion protein. These results also indicates that the α-galactosidase A fusion protein has excellent structural stability in the liquid formulation with a pH of 5.5 to 6.5.

### Experimental Example 2-5: Identification of Enzymatic Activity of α-Galactosidase A Fusion Protein

Biological activity of the α-galactosidase A fusion protein of the liquid formulation was identified in the same manner as in Example 3. Specifically, the liquid formulations #2 (pH 6.0) and #6 (pH 8.0) of Experimental Example 2-1 were analyzed by SE-HPLC analysis and pNP-Gal activity assay, such that the liquid formulation #2 (pH 6.0) was analyzed at the initial point of storage at 25° C. and the liquid formulation #6 (pH 8.0) was analyzed after 7 days of storage at 25° C.

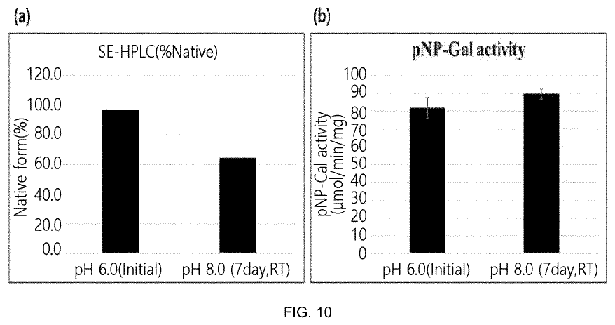

As a result, as shown in FIG. 10(a), in the SE-HPLC results, a percentage (%) of the native form of the liquid formulation at pH 6.0 at the initial point of storage was 96.7%, and a percentage (%) of the native form of the liquid formulation at pH 8.0 after 7 days of storage at room temperature was 64.0%. In addition, as shown in FIG. 10(b), the pNP-Gal activity exhibited a slight difference at an analysis deviation level (<20%). Based thereon, it was confirmed that although the purities of the α-galactosidase A fusion protein were slightly different between the two liquid formulations with the pH 6.0 and pH 8.0, the activity of α-galactosidase A was not significantly different.

These results indicate that although soluble aggregates are formed as some domains of α-galactosidase A are unfolded, the enzymatic activity may be retained.

### Experimental Example 3: Identification of Stability of α-Galactosidase a Fusion Protein According to Addition of Isotonic Agent to Liquid Formulation

### Experimental Example 3-1: Preparation of Formulation

In order to identify structural changes of the α-galactosidase A fusion protein according to the presence of an isotonic agent (NaCl) and concentration thereof, liquid formulations were prepared as follows.

TABLE 3

| | Concentration | | | |
|---|---|---|---|---|
| | of fusion | | | |
| # | protein | Buffer | Isotonic agent | pH |
| 1 | 10 mg/mL | 20 mM L-histidine | — | 6.0 |
| 2 | 10 mg/mL | 20 mM L-histidine | 50 mM sodium chloride | 6.0 |
| 3 | 10 mg/mL | 20 mM L-histidine | 100 mM sodium chloride | 6.0 |
| 4 | 10 mg/mL | 20 mM L-histidine | 200 mM sodium chloride | 6.0 |
| 5 | 10 mg/mL | 20 mM L-histidine | 300 mM sodium chloride | 6.0 |

### Experimental Example 3-2: SE-HPLC Analysis on Liquid Formulation According to Concentration of Isotonic Agent (Room Temperature Accelerated Stability)

Stability of various liquid formulations having different concentrations of the isotonic agent prepared in Experimental Example 3-1 were identified by a room temperature accelerated stability test. Specifically, the liquid formulations of Experimental Example 3-1 were analyzed by size exclusion liquid chromatography, all samples were analyzed at the initial point, after 7 days, and after 14 days of storage at 25° C.

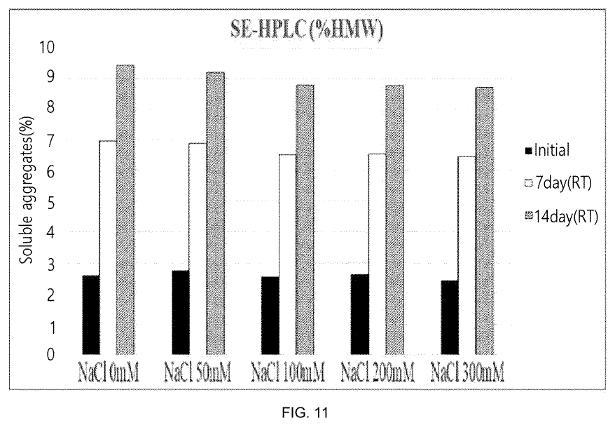

As a result, after two weeks of storage at room temperature, as the concentration of the isotonic agent (NaCl) increased from 0 mM to 50 mM, 100 mM, 200 mM, and 300 mM, it was confirmed that the ratios of the soluble aggregates (HMW) to the total fusion protein decreased by 9.4%, 9.2%, 8.8%, 8.8%, and 8.7%. In addition, there was no difference in the ratios of the soluble aggregates among the liquid formulations to which NaCl was added in an amount of 100 mM or more (FIG. 11).

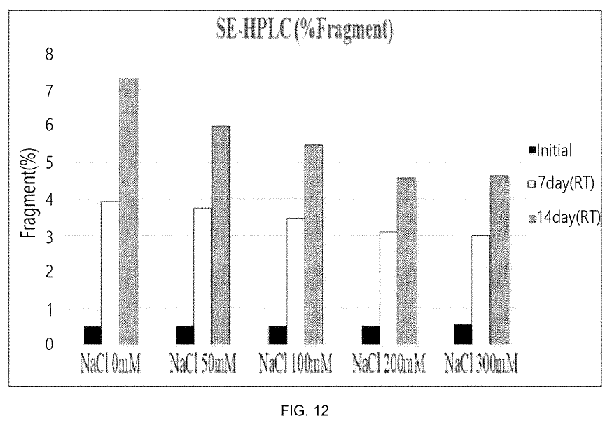

In addition, after two weeks of storage at room temperature, as the concentration of the isotonic agent (NaCl) increased from 0 mM to 50 mM, 100 mM, 200 mM, and 300 mM, the ratios of the fragments to the total fusion protein decreased by 7.3%, 6.0%, 5.5%, 4.6%, and 4.6%. In addition, there was no significant difference in the ratios of the fragments among the liquid formulations to which NaCl was added in an amount of 200 mM or more (FIG. 12).

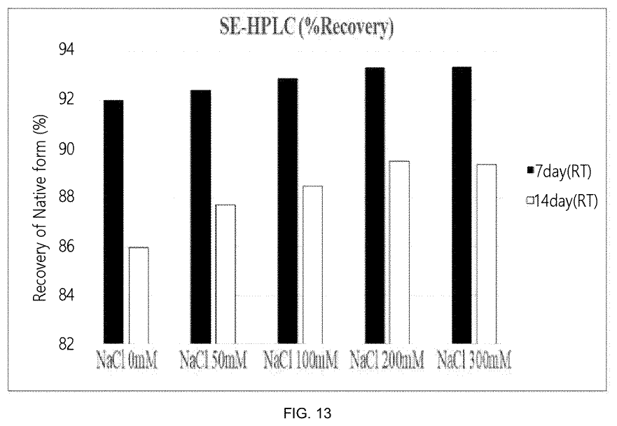

In addition, as a result of identifying the recovery of native form after two weeks of storage at room temperature, it was confirmed that the recovery of native form increased as the concentration of NaCl increased, and there was no significant difference in the recovery of native form among the liquid formulations to which NaCl was added in an amount of 200 mM or more (FIG. 13).

### Experimental Example 4: Identification of Stability of α-Galactosidase A Fusion Protein in Liquid Formulation According to Addition of Amino Acid

### Experimental Example 4-1: SE-HPLC Analysis on Liquid Formulation According to Type of Stabilizer (Amino Acid, Sugar, and Sugar Alcohol) (40° C. Accelerated Stability)

In order to identify structural change in α-galactosidase A fusion protein in the liquid formulation including a stabilizer (amino acid, sugar, or sugar alcohol) according to types of the stabilizer, liquid formulations were prepared as follows.

TABLE 4

| # | Concen- | | | | | |
|---|---|---|---|---|---|---|
| (amino acid, | tration | | | | | |
| sugar, sugar | of fusion | | | | | |
| alcohol, | protein | | Isotonic | Non-ionic | Stabi- | |
| 1.0% (w/v)) | pH | Buffer | agent | surfactant | lizer | |
| 1 | 10 | 20 mM | 50 mM | 0.05% | Arg | 6.0 |
| | mg/mL | L-histidine | sodium | polysor- | | |
| | | | chloride | bate 20 | | |
| 2 | 10 | 20 mM | 50 mM | 0.05% | Lys | 6.0 |
| | mg/mL | L-histidine | sodium | polysor- | | |
| | | | chloride | bate 20 | | |
| 3 | 10 | 20 mM | 50 mM | 0.05% | Ser | 6.0 |
| | mg/mL | L-histidine | sodium | polysor- | | |
| | | | chloride | bate 20 | | |
| 4 | 10 | 20 mM | 50 mM | 0.05% | Thr | 6.0 |
| | mg/mL | L-histidine | sodium | polysor- | | |
| | | | chloride | bate 20 | | |
| 5 | 10 | 20 mM | 50 mM | 0.05% | Asn | 6.0 |
| | mg/mL | L-histidine | sodium | polysor- | | |
| | | | chloride | bate 20 | | |
| 6 | 10 | 20 mM | 50 mM | 0.05% | Gln | 6.0 |
| | mg/mL | L-histidine | sodium | polysor- | | |
| | | | chloride | bate 20 | | |
| 7 | 10 | 20 mM | 50 mM | 0.05% | Gly | 6.0 |
| | mg/mL | L-histidine | sodium | polysor- | | |
| | | | chloride | bate 20 | | |
| 8 | 10 | 20 mM | 50 mM | 0.05% | Pro | 6.0 |
| | mg/mL | L-histidine | sodium | polysor- | | |
| | | | chloride | bate 20 | | |
| 9 | 10 | 20 mM | 50 mM | 0.05% | Ala | 6.0 |
| | mg/mL | L-histidine | sodium | polysor- | | |
| | | | chloride | bate 20 | | |
| 10 | 10 | 20 mM | 50 mM | 0.05% | Val | 6.0 |
| | mg/mL | L-histidine | sodium | polysor- | | |
| | | | chloride | bate 20 | | |
| 11 | 10 | 20 mM | 50 mM | 0.05% | Ile | 6.0 |
| | mg/mL | L-histidine | sodium | polysor- | | |
| | | | chloride | bate 20 | | |
| 12 | 10 | 20 mM | 50 mM | 0.05% | Leu | 6.0 |
| | mg/mL | L-histidine | sodium | polysor- | | |
| | | | chloride | bate 20 | | |
| 13 | 10 | 20 mM | 50 mM | 0.05% | Phe | 6.0 |
| | mg/mL | L-histidine | sodium | polysor- | | |
| | | | chloride | bate 20 | | |
| 14 | 10 | 20 mM | 50 mM | 0.05% | Su- | 6.0 |
| | mg/mL | L-histidine | sodium | polysor- | crose | |
| | | | chloride | bate 20 | | |
| 15 | 10 | 20 mM | 50 mM | 0.05% | Treha- | 6.0 |
| | mg/mL | L-histidine | sodium | polysor- | lose | |
| | | | chloride | bate 20 | | |

In order to identify stability of various liquid formulations having different types of stabilizers, an accelerated stability test was performed. Specifically, the liquid formulations of Table 4 were analyzed by size exclusion liquid chromatography, and all samples were analyzed after 15 hours of storage at 40° C.

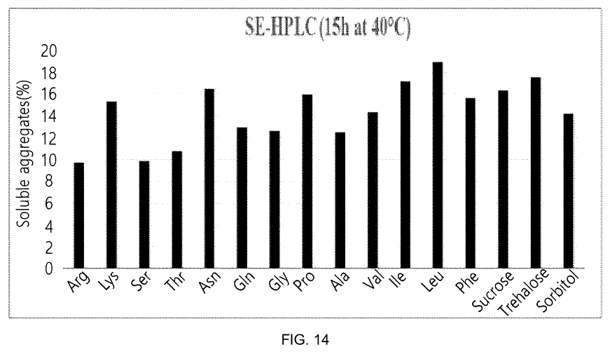

As a result, ratios of soluble aggregates of the liquid formulations #1 and #3 to which arginine and L-serine were added were 9.7% and 9.8%, respectively, which were lower than those of liquid formulations to which other types of amino acid were added (FIG. 14).

### Experimental Example 4-2: Tm Analysis According to Amino Acid Concentration

In order to analyze effects of concentrations of arginine and serine whose effects on stability of the liquid formulation were confirmed in Experimental Example 4-1, liquid formulations were prepared as follows.

TABLE 5

| | Concen- | | | | | |
|---|---|---|---|---|---|---|
| | tration | | | | | |
| | of fusion | | Isotonic | Non-ionic | | |
| # | protein | Buffer | agent | surfactant | Amino acid | pH |
| 1 | 10 | 20 mM | 50 mM | 0.05% | — | 5.5 |
| | mg/mL | | sodium | polysorbate | | |
| | | L-histidine | chloride | 20 | | |
| 2 | 10 | 20 mM | 200 mM | 0.05% | — | 5.5 |
| | mg/mL | | sodium | polysorbate | | |
| | | L-histidine | chloride | 20 | | |
| 3 | 10 | 20 mM | 50 mM | 0.05% | Arg 0.5% | 5.5 |
| | mg/mL | L-histidine | sodium | polysorbate | (w/v) | |
| | | | chloride | 20 | | |
| 4 | 10 | 20 mM | 50 mM | 0.05% | Arg 1.0% | 5.5 |
| | mg/mL | L-histidine | sodium | polysorbate | (w/v) | |
| | | | chloride | 20 | | |
| 5 | 10 | 20 mM | 50 mM | 0.05% | Arg 2.0% | 5.5 |
| | mg/mL | L-histidine | sodium | polysorbate | (w/v) | |
| | | | chloride | 20 | | |
| 6 | 10 | 20 mM | 50 mM | 0.05% | Arg 4.0% | 5.5 |
| | mg/mL | L-histidine | sodium | polysorbate | (w/v) | |
| | | | chloride | 20 | | |
| 7 | 10 | 20 mM | 50 mM | 0.05% | Ser 0.5% | 5.5 |
| | mg/mL | L-histidine | sodium | polysorbate | (w/v) | |
| | | | chloride | 20 | | |
| 8 | 10 | 20 mM | 50 mM | 0.05% | Ser 1.0% | 5.5 |
| | mg/mL | L-histidine | sodium | polysorbate | (w/v) | |
| | | | chloride | 20 | | |
| 9 | 10 | 20 mM | 50 mM | 0.05% | Ser 2.0% | 5.5 |
| | mg/mL | L-histidine | sodium | polysorbate | (w/v) | |
| | | | chloride | 20 | | |
| 10 | 10 | 20 mM | 50 mM | 0.05% | Ser 4.0% | 5.5 |
| | mg/mL | L-histidine | sodium | polysorbate | (w/v) | |
| | | | chloride | 20 | | |
| 11 | 10 | 20 mM | 50 mM | 0.05% | Arg 1.0% | 5.5 |
| | mg/mL | L-histidine | sodium | polysorbate | (w/v) + Ser | |
| | | | chloride | 20 | 1.0% (w/v) | |
| 12 | 10 | 20 mM | 50 mM | 0.05% | Arg 2.0% | 5.5 |
| | mg/mL | L-histidine | sodium | polysorbate | (w/v) + Ser | |
| | | | chloride | 20 | 2.0% (w/v) | |

Tm values of the liquid formulations #1 to #12 of Table 5 were measured using an UNCLE device of Example 2.

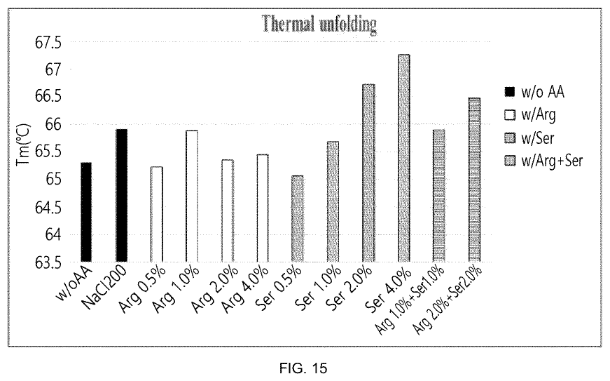

5 As a result, while the Tm value increased in proportion to the concentration of serine, the relevance between the concentration of arginine and the Tm value was not confirmed. In addition, lower Tm values were obtained in the liquid formulations to which arginine was added at concentrations of 2.0% and 4.0% than the liquid formulations to which serine was added at concentrations of 2.0% and 4.0%. Furthermore, it was confirmed that a synergistic effect was not obtained even when a mixture of serine and arginine was used as a stabilizer (FIG. 15).

### Experimental Example 4-3: SE-HPLC Analysis on Liquid Formulation According to Amino Acid Concentration (Room Temperature Accelerated Stability)

Stability of liquid formulations containing arginine and serine according to amino acid concentration was identified by a room temperature accelerated stability test.

Specifically, room temperature accelerated stability was measured in the liquid formulations including various concentrations of serine and arginine as shown in Table 5 by measuring soluble aggregate formation rates and recovery of native form. The liquid formulations of Table 5 were analyzed by size exclusion liquid chromatography, and all of the liquid formulations were analyzed at the initial point, after 1 day, after 4 days, and after 7 days of storage at 25° C.

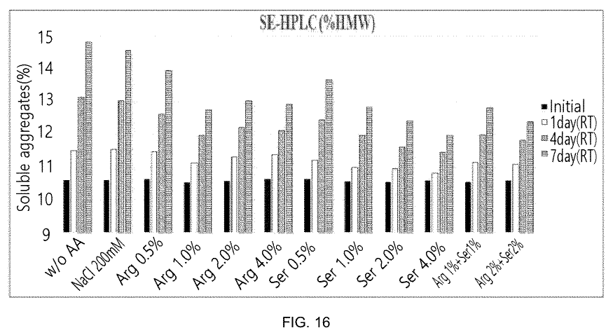

As a result, in the case of adding serine, it was confirmed that the soluble aggregate formation rates decreased as the concentration of serine increase with the lapse of storage time, particularly, the effect on inhibiting formation of soluble aggregates was excellent even when serine was added in a small amount of 0.5% (w/v) compared to that of the control (liquid formulations #1 and #2). In addition, it was also confirmed that serine exhibited superior effects on inhibiting formation of soluble aggregates to the liquid formulation including arginine as a stabilizer. In the same manner as in FIG. 15, the stability in the case of adding a mixture of arginine and serine was not significantly different from the stability in the case of adding serine alone (FIG. 16).

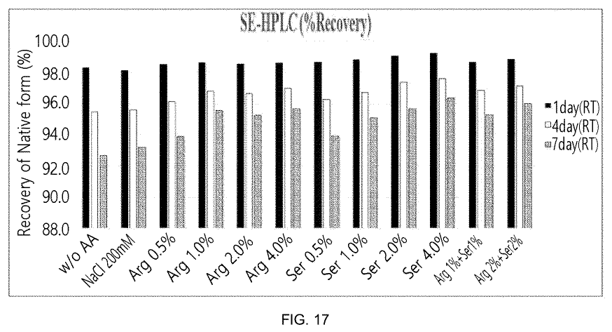

In addition, it was confirmed that the recovery of native form increased in the liquid formulation to which serine was added as the concentration of the added serine increased, particularly the recovery of native form was superior to that of the control (liquid formulations #1 and #2) even when serine was added in a small amount of 0.5% (w/v). Also, it was confirmed that serine was superior to maintain the native form compared to liquid formulations containing arginine as a stabilizer. In the same manner as in FIG. 15, the stability in the case of adding a mixture of arginine and serine was not significantly different from the stability in the case of adding serine alone (FIG. 17).

These results indicate that by adding an amino acid, particularly, serine, the structural stability of the α-galactosidase A fusion protein may be improved to prevent unfolding of α-galactosidase A and formation of soluble aggregates may be inhibited as much as possible to increase stability of the liquid formulation.

### Experimental Example 5: Identification of Stability of High-Concentration α-Galactosidase A Fusion Protein

Based on the composition of the liquid formulation whose stability was confirmed in the above-described experimental examples, in order to identify whether stability of the liquid formulation is maintained even when the α-galactosidase A fusion protein is contained at a high concentration, an experiment was performed as follows.

### Experimental Example 5-1: Identification of Stability of High-Concentration (50 mg/mL) α-Galactosidase A Fusion Protein (Room Temperature Accelerated Stability)

Liquid formulations were prepared as follows to identify stability of a liquid formulations including the α-galactosidase A fusion protein at a concentration of 50 mg/mL.

TABLE 6

| | Concen- | | | | | |
|---|---|---|---|---|---|---|
| | tration | | | | | |
| | of fusion | | Isotonic | Non-ionic | Amino | |
| # | protein | Buffer | agent | surfactant | acid | pH |
| 1 | 50 | 20 mM | 50 mM | 0.05% | — | 5.5 |
| | mg/mL | L-histidine | sodium | polysorbate | | |
| | | | chloride | 20 | | |
| 2 | 50 | 20 mM | 200 mM | 0.05% | — | 5.5 |
| | mg/mL | L-histidine | sodium | polysorbate | | |
| | | | chloride | 20 | | |
| 3 | 50 | 20 mM | 50 mM | 0.05% | Ser 0.5% | 5.5 |
| | mg/mL | L-histidine | sodium | polysorbate | (w/v) | |
| | | | chloride | 20 | | |
| 4 | 50 | 20 mM | 50 mM | 0.05% | Ser 1.0% | 5.5 |
| | mg/mL | L-histidine | sodium | polysorbate | (w/v) | |
| | | | chloride | 20 | | |
| 5 | 50 | 20 mM | 50 mM | 0.05% | Ser 2.0% | 5.5 |
| | mg/mL | L-histidine | sodium | polysorbate | (w/v) | |
| | | | chloride | 20 | | |
| 6 | 50 | 20 mM | 50 mM | 0.05% | Ser 4.0% | 5.5 |
| | mg/mL | L-histidine | sodium | polysorbate | (w/v) | |
| | | | chloride | 20 | | |

The liquid formulations prepared as shown in Table 6 were analyzed by size exclusion liquid chromatography, and all liquid formulations were analyzed at the initial point, after 1 day, after 4 days, and after 7 days of storage at 25° C.

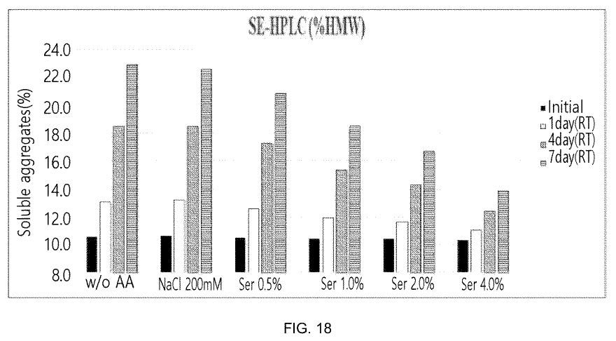

As a result, it was confirmed as the concentration of serine increased, the soluble aggregate (HMW) formation rates relative to the total fusion protein decreased with the lapse of storage time, particularly, the effect on inhibiting formation of soluble aggregates was superior even when serine was added in a small amount of 0.5% (w/v) compared to that of the control (liquid formulations #1 and #2) (FIG. 18).

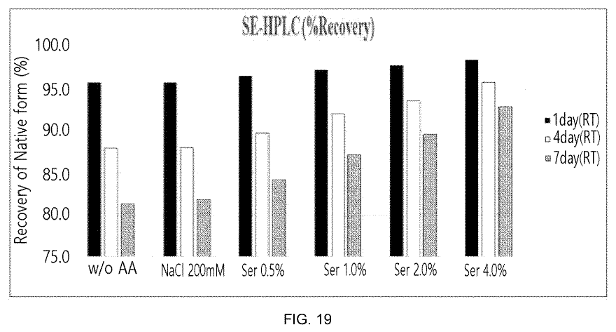

In addition, it was confirmed that the recovery of native form increased as the concentration of serine increased, and the higher recovery of native form was obtained even when serine was added in a small amount of 0.5% (w/v) compared to that of the control (liquid formulations #1 and #2) (FIG. 19).

The effect of serine on inhibiting formation of soluble aggregates was superior in the liquid formulation containing the fusion protein at a concentration of 50 mg/mL compared to the effect in the liquid formulation containing the fusion protein at a concentration of 10 mg/mL, indicating that serine is important in the liquid formulation containing the fusion protein at a high-concentration.

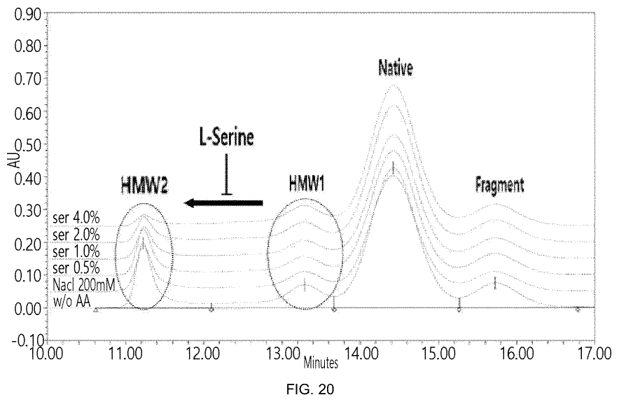

In addition, as a result of analyzing SE-HPLC chromatogram after 7 days of storage at 25° C., it was confirmed that the progress from soluble aggregates (HWM1) to higher-order aggregates (HMW2) was significantly inhibited as the concentration of serine increased (FIG. 20). The higher-order aggregates are aggregates formed as the unfolding of the soluble aggregates proceeds. As higher-order aggregates are formed and the size of aggregates increases, the risk of immunogenicity increases, and thus safety of the liquid formulation may be increased by inhibiting formation of the higher-order aggregates by adding serine.

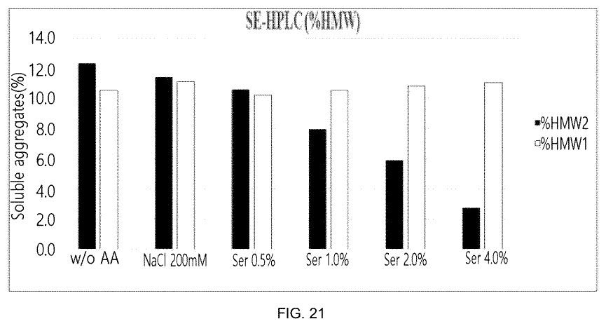

Similarly, as the concentration of serine increased, formation of higher-order aggregates (HMW2) significantly decreased compared to formation of soluble aggregates (HMW1) (FIG. 21).

### Experimental Example 5-2: Identification of Stability of High-Concentration (90 mg/mL) α-Galactosidase A Fusion Protein (Stability During 3-Week Cold Storage)

In order to identify stability of liquid formulations including the α-galactosidase A fusion protein at a high concentration (90 mg/mL) compared to that of Experimental Example 5-1, liquid formulations were prepared as follows.

TABLE 7

| | Concen- | | | | | |
|---|---|---|---|---|---|---|
| | tration | | | | | |
| | of fusion | | Isotonic | Non-ionic | Amino | |
| # | protein | Buffer | agent | surfactant | acid | pH |
| 1 | 90 | 20 mM | 150 mM | 0.05% | — | 6.0 |
| | mg/mL | L-histidine | sodium | polysorbate | | |
| | | | chloride | 20 | | |
| 2 | 90 | 20 mM | 50 mM | 0.05% | Ser 2.0% | 6.0 |
| | mg/mL | L-histidine | sodium | polysorbate | (w/v) | |
| | | | chloride | 20 | | |

In order to measure appearance and turbidity of the formulations shown in Table 7, visual inspection and UV spectroscopy were used. The analysis was performed at an initial point and after 21 days of storage at 2° C. to 8° C.

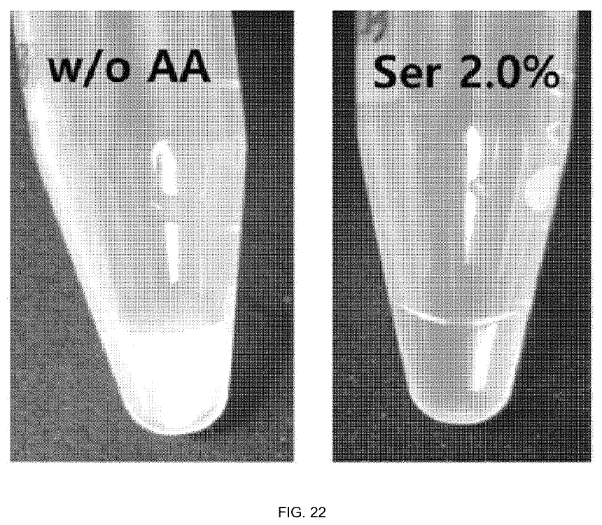

As a result of comparing appearance of liquid formulations including a high-concentration (90 mg/mL) of the fusion protein, the liquid formulation #1 not including an amino acid as a stabilizer had turbid appearance, while the liquid formulation #2 containing 2.0% (w/v) serine had a clear and transparent appearance (FIG. 22).

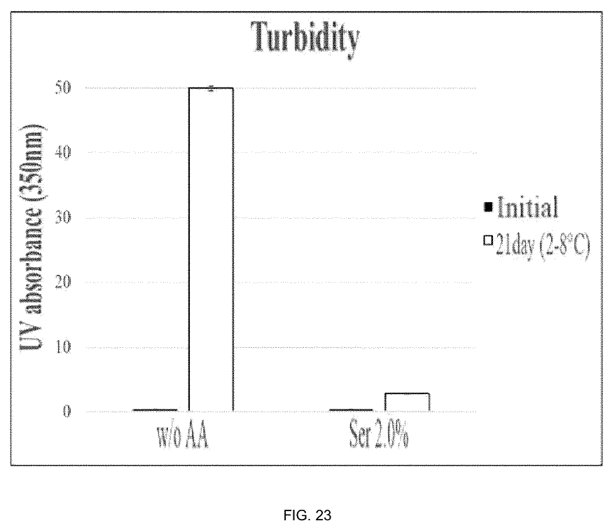

In addition, as a result of comparing turbidity, the turbidity of the liquid formulation #1 not including an amino acid as a stabilizer was 49.9 and the turbidity of the liquid formulation #2 including 2.0% (w/v) serine was 2.8. Based thereon, it was confirmed that turbidity of the liquid formulation was reduced in the case of including the amino acid (serine) as the stabilizer, and thus stability of the liquid formulation was improved (FIG. 23).

These results indicate that the liquid formulation of the present invention has stability even when the α-galactosidase A fusion protein is contained at a high concentration.

The above description of the present invention is provided for the purpose of illustration, and it would be understood by those skilled in the art that various changes and modifications may be made without changing technical conception and essential features of the present invention. Thus, it is clear that the above-described embodiments are illustrative in all aspects and do not limit the present invention. Furthermore, the scope of the present invention should be defined by the appended claims rather than the detailed description, and it should be understood that all modifications or variations derived from the meanings and scope of the present invention and equivalents thereof are included in the scope of the present invention.

1. A liquid formulation comprising a fusion protein in which α-galactosidase A is fused to an immunoglobulin Fc region, the liquid formulation comprising:
the fusion protein at a concentration of 10 mg/mL to 90 mg/ml;
a buffer for maintaining a pH of the liquid formulation in a range of 5.5 to 6.5; and
an amino acid at a concentration of 0.5% (w/v) to 4.0% (w/v).

2. The liquid formulation according to claim 1, wherein the buffer comprises histidine or a salt thereof, citric acid or a salt thereof, acetic acid or a salt thereof, phosphoric acid or a salt thereof, or any combination thereof.

3. The liquid formulation according to claim 1, further comprising an isotonic agent at a concentration of 5 mM to 200 mM.

4. The liquid formulation according to claim 3, wherein the isotonic agent is sodium chloride.

5. The liquid formulation according to claim 1, wherein the amino acid is selected from the group consisting of arginine, serine, threonine, glutamine, glycine, alanine, and any combination thereof.

6. The liquid formulation according to claim 1, further comprising a non-ionic surfactant at a concentration of 0.005% (w/v) to 0.1% (w/v).

7. The liquid formulation according to claim 6, wherein the non-ionic surfactant is selected from poloxamer 188, polysorbate 20, polysorbate 40, polysorbate 60, polysorbate 80, and any combination thereof.

8. The liquid formulation according to claim 1, wherein the α-galactosidase A comprises an amino acid sequence of SEQ ID NO: 1.

9. The liquid formulation according to claim 1, wherein the fusion protein comprises an amino acid sequence of SEQ ID NO: 4.

10. The liquid formulation according to claim 1, wherein the fusion protein has a structure in which two molecules of α-galactosidase A are linked to each monomer of an immunoglobulin Fc region in a dimeric form.

11. The liquid formulation according to claim 1, wherein the liquid formulation comprises:
the fusion protein at a concentration of 10 mg/ml to 90 mg/ml;
10 mM to 50 mM histidine; and
1.0% (w/v) to 4.0% (w/v) serine.

12. The liquid formulation according to claim 1, wherein the liquid formulation is used to prevent or treat Fabry disease.

13. The liquid formulation according to claim 1, wherein the liquid formulation is administered via a subcutaneous route.


SEQUENCE LISTING (5 sequences)

SEQ ID NO: 1 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 2 - protein, 30 aa, synthetic construct
  REGION 1..30: note = peptide linker
GGGGSGGGGSGGGGSGGGGSGGGGSGGGGS

SEQ ID NO: 3 - protein, 221 aa, synthetic construct
  REGION 1..221: note = Fc varinat
PPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 680 aa, synthetic construct
  REGION 1..680: note = alpha galactosidase-Fc fusion protein
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLLGGGGSGGGGSGGGGSGGGGSGGGGSGGGGSPPCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - DNA, 2043 bp, synthetic construct
  misc_feature 1..2043: note = alpha galactosidase-Fc fusion protein
  source 1..2043: mol_type = other DNA
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttacttggcggcggaggttcaggtggtggtggctctggcggtggagggtcggggggaggcggctctggaggagggggctccggtgggggaggtagcccaccatgcccagcacctgagttcctggggggaccatcagtcttcctgttccccccaaaacccaaggacaccctcatgatctcccggacccctgaggtcacatgcgtggtggtggacgtgagccaggaagaccctgaggtccagttcaactggtacgtggacggcgtggaggtgcataatgccaagacaaagccgcgggaggagcagttccaaagcacgtaccgtgtggtcagcgtcctcaccgtcctgcaccaggactggctgaatggcaaggagtacaagtgcaaggtctccaacaaaggcctcccatcctccatcgagaaaaccatctccaaagccaaagggcagccccgagaaccacaggtgtacaccctgcccccatcccaggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaggctaaccgtggacaagagcaggtggcaggaggggaacgtcttctcatgctccgtgatgcatgaggctctgcacaaccactacacgcagaagagcctctccctgtctctgggtaaatga